(19) United States
(12) Patent Application Publication
(10) Pub. No.: US 2003/0017518 A1
(43) Pub. Date: Jan. 23, 2003

(54) Non-radio-active assay of LPS kinases

(76) Inventors: Joseph Lam, Guelph (CA); Xin Zhao, Beijing (CN)

Correspondence Address: KRAMER + ASSOCIATES, P.C., CRYSTAL PLAZA ONE, 2001 JEFFERSON DAVIS HWY. SUITE 1101, ARLINGTON, VA, 22202, US

(21) Appl. No.: 10/179,851

(22) Filed: Jun. 26, 2002

Related U.S. Application Data
(60) Provisional application No. 60/300,420, filed on Jun. 26, 2001.

(51) Int. Cl. G01N 33/554; G01N 33/569

(52) U.S. Cl. 435/7.32

(57) ABSTRACT

The present invention involves a method of assaying for modulators of enzymes involved in the phosphorylation of the inner core oligosaccharide of LPS. In particular, the method assays for modulators, preferably inhibitors, of WaaP, a tyrosine kinase responsible for the phosphorylation of HepI of the inner core LPS. The finding that monoclonal antibody mAb 7-4 specifically recognizes the phosphate group(s) of LPS, is the basis for the development of a non-radiolabeling, ELISA-based assay for enzymes involved in the phosphorylation of LPS.

### CROSS REFERENCE TO RELATED APPLICATION

This application claims priority UNDER 35 USC 119(e) from U.S. provisional application No. 60/300,420 filed on Jun. 26, 2001, which is incorporated by reference herein in its entirety.

### FIELD OF THE INVENTION

The present invention relates generally to protein assays. More specifically, the present invention relates to the assay of enzymes involved in the phosphorylation of the oligosaccharide of lipopolysaccharide (LPS) in bacteria.

### BACKGROUND OF THE INVENTION

Pseudomonas aeruginosa is an opportunistic pathogen that can cause bacterial infections in compromised patients including those receiving chemotherapy, or suffering burn wounds or cystic fibrosis. LPS located in the outer membrane of P. aeruginosa is one of the major virulent factors. It is composed of lipid A, core oligosaccharide (including outer core and inner core regions) and O antigen (FIG. 1). The inner core of LPS is composed of L-glycero-D-manno-heptose (Hep) and 3-deoxy-D-manno-octulosonic acid (Kdo). The LPS of P. aeruginosa is known to be the most highly phosphorylated among Gram-negative bacteria (Sadovskaya, 1998). The multiple phosphoryl substituents in this region are essential for the outer membrane stability (Walsh, 2000). Its inner core possesses three phosphate groups on C2, C4 and C6 of HepI (FIG. 1) respectively. These phosphate substituents contribute negative charges that are crucial in forming ionic bridge with divalent cations to stabilize the outer membrane.

waaP, involved in the phosphorylation of HepI of P. aeruginosa LPS, has been investigated at the genetic level (Walsh, 2000). Mutation of this gene was lethal to the bacterium, and the knockout of the chromosomal waaP gene was accomplished only when another copy of waaP was added in trans (Walsh, 2000). Thus, it was established that the presence of phosphate(s) on the HepI is essential for the viability of P. aeruginosa. Furthermore, waaP_{P. aeruginosa} (waaP_{Pa}) can complement the waaP mutant in Salmonella typhimurium to restore the resistance to SDS and novobiocin in this mutant. It was also demonstrated that waaP can reconstitute the phosphate on C4 of HepI by 2D ¹H/³¹P-NMR analysis. These data enabled the determination that waaP putatively encodes a sugar kinase to phosphorylate C4 on HepI (Walsh, 2000). More importantly, since WaaP is crucial to P. aeruginosa, its inhibitors have therapeutic value. Therefore, this protein is an attractive target for the development of novel drugs to control the infection from P. aeruginosa which is intrinsically resistant to a wide range of antibiotics. This requires an in-depth understanding of the biochemical properties of this enzyme and development of an assay that can be automated for screening large numbers of potential inhibitors.

In eukaryotes, protein tyrosine kinases have been well characterized and identified to play important functions in biological regulation i.e. signal transduction and growth control. The crystallization of many of these protein kinases has provided the insight into the molecular recognition at the substrate and ATP binding sites as well as the mechanisms of action of these enzymes. However, not much is known at present about the tyrosine kinases in prokaryotes as they are regarded as rare and are poorly defined (Ilan, 1999; Zhang, 1996 and Cozzone, 1998). A few recent reports described the phosphotryrosine-kinases involved in polysaccharide biosynthesis. These kinases include Wzc_{cps} in Escherichia coli with group 1 capsules (Wugeditsch, 2000), Wzc_{ca} in E. coli K-12 (Vincent, 1999; 2000), Etk in E. coli (Ilan, 1999), Ptk in Acinetobacter johnosoii (Duclos, 1996; Grangeasse, 1998) and CpsD in Streptococcus pneunioniae (Morona, 2000). Most of these enzymes are either proposed or identified to be involved in the transportation or regulation of the production of exopolysaccharides required for virulence (Grangeasse, 1998; Ilan, 1999). Interestingly, none of them showed significant homology to the typical tyrosine kinases from eukaryotes (Hanks, 1991). Thus far, no protein tyrosine kinase has been reported to phosphorylate the sugar residue in the lipopolysaccharide of Gram-negative bacteria.

In E. coli the kinase activity of WaaP (WaaP_{Ec}), which shared 52% homology with WaaP_{Pa}, has been demonstrated by an assay using [³³P]ATP to phosphorylate the LPS from the waaP knockout mutant of E. coli (Yelthon, 2001). In that study, the authors focused on the purification of the enzyme and characterization of the enzyme kinetics, but they did not investigate whether WaaP was a tyrosine kinase or not.

In general, previous methods for the measurement of kinase activities use [³²P] or [³³P]ATP and the substrates. The incorporation of [³²P] or [³³P]-phosphate is detected by precipitating the polypeptide substrate on filter discs with trichloroacetic acid, extensively washing, and counting for radioactivity by conventional liquid scintillation methods (Schraag, 1993). These steps are tedious, difficult to automate and labor-intensive when performed with a large number of samples (Braunwalder, 1996). There remains a need for a simpler method for measuring the activity of WaaP and its homologs. Preferably the method should not require the use of radiolabeled substrates and should be amenable to automation.

### SUMMARY OF THE INVENTION

WaaP has been overexpressed, purified and characterized as an autophosphorylated tyrosine kinase that is essential for the phosphorylation of the HepI residue in P. aeruginosa (Zhao and Lam, 2002). This is the first report of a sugar kinase in prokaryotes that showed features that are shared among the eukaryotic-type protein-tyrosine kinases. An enzyme-linked immunosorbent assay (ELISA) has been developed for the determination of the enzyme activity of WaaP to phosphorylate LPS. HF-LPS, the dephosphorylated LPS obtained by hydrofluoric acid (HF) treatment, was generated, characterized and used as the substrate in the enzyme assay. A monoclonal antibody (mAb) 7-4, is specific for the inner core oligosaccharide of P. aeruginosa (de Kievit and Lam, 1994). This antibody is useful for detection of bacteria having LPS and is useful in determining whether or not WaaP is phosphorylated. Consequently it is useful in an assay to determine the enzymatic activity of WaaP and other enzymes involved in the phosphorylation of the inner core oligosaccharide of LPS. Antibody 7-4 specifically recognizes the phosphate group(s) on LPS and therefore, is the primary antibody in the ELISA.

A method for assaying for modulators of an enzyme involved in the phosphorylation of the inner core oligosaccharide of lipopolysaccharide (LPS), comprising the steps of:

(a) incubating a test sample comprising (i) the enzyme, (ii) a candidate substance; and (iii) substrates comprising dephosphorylated LPS and a source of phosphate;

(b) adding to the test sample at least one antibody comprising an antibody that binds to phosphorylated LPS while not binding to dephosphorylated LPS; and

(c) detecting phosphorylated LPS in the test sample by measuring the binding of the at least one antibody to phosphorylated LPS, wherein an increase or decrease in the amount of phosphorylated LPS in the test sample in the presence of the candidate substance indicates that the substance is a modulator. The reaction is preferably stopped prior to the detection step. One preferably determines the presence of phosphorylated LPS in the test sample by detecting the binding to LPS, wherein the presence of phosphorylated LPS indicates that the substance is a modulator. In one variation, the detected phosphoryulated LPS is. quantified by determining the amount of phosphorylated LPS in the test sample by measuring the binding of the at least one antibody to phosphorylated LPS, wherein a change in the amount of phosphorylated LPS in the test sample compared to an amount of phosphorylated LPS in a control sample (that does not contain the substance suspected of being a modulator) indicates that the substance is a modulator. Instead of using control samples, the detected phosphorylated LPS may be quantified by other means or compared to known levels of phosphorylated LPS determined from prior assays or experience.

In preferred embodiments of the invention the antibody that binds to phosphorylated LPS while not binding to dephosphorylated LPS is mAb 7-4. Preferably, the assay is used to screen for inhibitors of an enzyme involved in the phosphorylation of the inner core oligosaccharide of LPS. A method for assaying for inhibitors of an enzyme involved in the phosphorylation of the inner core oligosaccharide of LPS, comprising the steps of:

(a) incubating a test sample comprising (i) the enzyme, (ii) a candidate substance; and (iii) substrates comprising dephosphorylated LPS and a source of phosphate;

(b) adding to the test sample at least one antibody that binds to phosphorylated LPS while not binding to dephosphorylated LPS; and

(c) detecting phosphorylated LPS in the test sample by measuring the binding of the at least one antibody to phosphorylated LPS, wherein a decrease in the amount of phosphorylated LPS in the test sample in the presence of the candidate substance indicates that the candidate substance is an inhibitor.

One preferably determines the presence of phosphorylated LPS in the test sample by detecting the binding to LPS, wherein the presence of phosphorylated LPS indicates that the substance is a modulator. The amount of phosporylated LPS may be quantified as described above with respect to modulators. One may measure the binding of the at least one antibody to phosphorylated LPS, wherein a decrease in the amount of phosphorylated LPS in the test sample compared to an amount of phosphorylated LPS in a control sample (that does not contain the substance suspected of being an inhibitor) indicates that the substance is an inhibitor. The reaction is preferably stopped prior to the detection step.

In preferred embodiments of the invention, the antibody that binds to phosphorylated LPS while not binding to dephosphorylated LPS is mAb 7-4.

The method of the invention can be used for the screening of novel antimicrobial compounds against infection from P. aeruginosa and a host of other bacteria, preferably Gram-nagative bacteria.

The present invention also includes kits to perform the method of the invention comprising:

(a) reagents for performing an enzyme reaction, including an aliquot of dephosphorylated LPS, an aliquot of a source of phosphate, and an aliquot of an enzyme involved in the phosphorylation of the inner core oligosaccharide of LPS; and

(b) reagents for performing an ELISA, including an aliquot of an antibody that binds to phosphorylated LPS while not binding to dephosphorylated LPS and an aliquot of the secondary antibody.

Preferably the antibody that binds to phosphorylated LPS while not binding to dephosphorylated LPS is mAb 7-4.

The invention includes a modulator or inhibitor identified with a method of the invention.

Another aspect of the present invention involves a method of conducting a target discovery business using the method of the invention.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

Embodiments of the invention will now be described in relation to the drawings in which:

FIG. 1 is a schematic showing the chemical structure of the core oligosaccharide region of lipopolysaccharide of Pseudomonas aeruginosa serotype O5 (PAO1). Glc, D-glucose; Gal, D-galactose; Hep, L-glycero-D-manno-heptose; Rha, L-rhamnose; Ala, L-alanine; GaIN, D-galactosamine; Kdo, 3-deoxy-D-manno-octulosonic acid; P, phosphate group;

FIG. 2 is a schematic showing the alignment analysis of the amino acid sequence of WaaP from P. aeruginosa with WaaPE. coli and protein kinases from eukaryotic cells. The subdomains I-IX were defined based on the nomenclature of Hanks (Hanks, 1991) PKC-α: protein kinase C, α form from bovine brain (Parker, 1986); SNF1: “sucrose nonfermenting” mutant wild-type gene product from Sacchromyces cerevisiae (Celenza, 1986); Src: cellular homolog of oncogene product from Rous avian sarcoma virus from human fetal liver (Anderson, 1985); EGFR: epidermal growth factor receptor from human placenta and A431 cell line (Ullrich, 1984). Conserved functional amino acids were labeled in dark background;

FIG. 3 is a SDS-PAGE gel (identified by Coommassie Blue R250 staining) depicting the results of the overexpression and purification of recombinant WaaPHisC. Protein samples were loaded on 12.5% SDS-PAGE gel and identified by Coommassie Blue R250 staining (A) and Western immunoblotting with Penta-His antibody (B). Lane 1: SeeBlue Pre-Stained Standards; Lane 2: Induced Vector pET30a/E. coli BL21(DE3)pLysS without waaP insert; Lane 3, 4: overexpression of WaaPHisC-pET30a/E. coli BL21(DE3)pLysS pre- (Lane 3) and post- (Lane 4) induction with 1 mM IPTG; Lane5: IMAC purification of WaaPHisC.;

FIG. 4 is a Western immunoblot showing WaaPHisC with anti-phosphotyrosine kinase mAb PY-20 as the primary antibody. Protein was transferred from 12.5% SDS-gel onto PVDF membrane for Western immunoblotting. 1: SeeBlue Pre-Stained Standards; 2: WaaPHisC purified by IMAC.;

FIG. 5 is a plot from the analysis of WaapHisC using Matrix-assisted laser desorption/ionization time-of-flight (MALDI-TOF) mass spectrometry. WaaPHisC was purified by IMAC. The actual mass of WaaPHisC was m/z 33544.618 comparing to the predicted mass (without phosphate groups) of 32897.38. The extra mass of 647.328 corresponded to eight phosphate groups.;

FIG. 6A is a silver-stained SDS-PAGE showing the characterization of HF-LPS in comparison with PAO1-LPS. Characterization of HF-LPS with SDS-PAGE and different LPS monoclonal antibodies in the comparison with PAO1-LPS. A:,silver stained SDS-PAGE gel; Western immunoblotting with monoclonal antibodies;

FIG. 6B is a Western immunoblot showing the identification of HF-LPS and PAO1-LPS with inner core-specific monoclonal antibody mAb 7-4;

FIG. 6C is a Western immunoblot showing the identification of HF-LPS and PAO1-LPS with B-band-specific monoclonal antibody, MF15-4;

FIG. 6D is a Western immunoblot showing the identification of HF-LPS and PAO1-LPS with A-band-specific monoclonal antibody, NF10;

FIG. 6E is a Western immunoblot showing the identification of HF-LPS and PAO1-LPS with outer core-specific monoclonal antibody, 5c101;

FIG. 6F is a Western immunoblot showing the identification of HF-LPS and PAO1-LPS with semi-rough core-specific monoclonal antibody 18-19;

FIG. 7 is a graph showing the determination of the critical aggregation concentrations (CAC) of PAO1-LPS and HF-LPS. Analysis was performed in 50 μl solution containing 20 mM, Tris-HCl, 150 mM NaCl, pH 7.5 in the presence of 5 μM N-phenyl-1-naphthylamine (NPN). The fluorescence was measured with excitation wavelength at 350 nm, emission at 425 nm. The CAC can be determined where the lines of the two slopes (a and b) of each curve intercept (the cross points with line c), the x-value of point A for PAO1-LPS and point B for HF-LPS;

FIG. 8 is a graph showing the phosphate analysis on PAO1-LPS and HF-LPS. K₂HPO₄ was used as the standard (inlet);

FIG. 9A is a graph showing the ELISA developed with colorimetric method by reading A_{405 nm};

FIG. 9B is a graph showing the ELISA developed with chemiluminescence method with CDP*;

FIG. 10 is a graph showing the comparison of chemiluminescence responses from the ELISAs of PAO1-LPS using simultaneous vs consecutive incubation of primary and secondary antibody;

FIG. 11 is a graph showing the comparison of chemiluminescence responses from the ELISAs of PAO1-LPS using different dilutions of CDP* (at without dilution, 1:1, 1:3, 1:5, 1:7, 1:9 and 1:11 dilutions) for the developing step;

FIG. 12 is a graph showing the recognition of PAO1-LPS and HF-LPS by mAb 7-4 monoclonal antibody in ELISA (chemiluminescence response);

FIG. 13A is a graph showing the time course of the phosphate reconstitution of HF-LPS by WaaPHisC using ELISA (chemiluminescence response). The reactions were performed in 50 μl solution in triplicate and then coated on the 96-well microtiter plate for ELISA.;

FIG. 13B is a graph showing the ELISA chemiluminescence response of the phosphate reconstitution of HF-LPS by WaaPHisC using different amounts of enzyme;

FIG. 13C is a graph showing the ELISA chemiluminescence response of the phosphate reconstitution of HF-LPS by WaaPHisC using different amounts of ATP; and

FIG. 13D is a graph showing the ELISA chemiluminescence response of the phosphate reconstitution of HF-LPS by WaaPHisC using different amounts of HF-LPS.

FIG. 14A is the nucleotide sequence of the 7-4 heavy chain immunoglobulin gene (SEQ ID NO: 1);

FIG. 14B is the amino acid sequence of the 7-4 heavy chain immunoglobulin gene (SEQ ID NO: 2);

FIG. 14C shows nucleotide sequences of the heavy chain V regions for O25G3D6 and 7-4 aligned with their germ-line gene, H10. Dashes represent identity with the representative germ-line sequence and base and amino acid substitutions are shown with the appropriate lettering. Solid lines indicate CDR's. Gaps, represented by dots in V_{H}7-4, have been introduced into the CDR to facilitate alignment among the sequences. The O25G3D6, 74 and 177V_{H} sequences are available from Genebank under accession numbers U2599, U25100 and U25102, respectively. Key: CDR=complementarity determining region (or hypervariable region); V_{H}H10=variable heavy chain of germ line gene H10, in which 7-4 is highly homologous and a member of this gene family; V_{H}O25G3D6=variable heavy chain of a different monoclonal antibody, specific for B-band O-antigen of serotype O6.

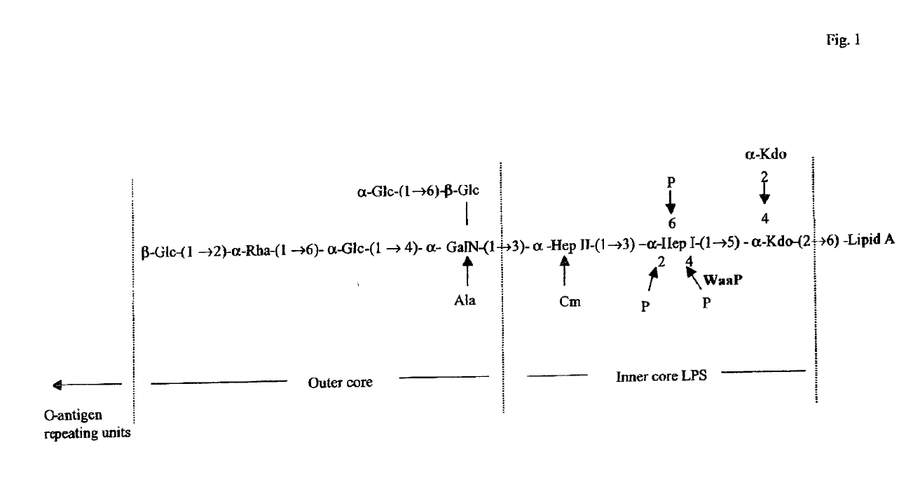

### DETAILED DESCRIPTION OF THE INVENTION

As used herein, the following symbols have the following meaning: LPS, lipopolysaccharide; UDP, uridyl diphospho nucleotide; Glc, glucose; Gal, galactose; GlcNAc, N-acetyl glucosamine; GalNAc, N-acetyl galactosanmine; CE, capillary electrophoresis; PAGE, polyacrylamide gel electrophoresis. IPTG, isopropyl-1-thio-β-D galactopyranoside; IMAC, immobilized metal affinity chromatography.

### Antibodies

Antibodies to LPS may be prepared as described in this application. The term “antibody” as used herein is intended to include fragments thereof which also specifically react with LPS, or fragments thereof. Fragments include F(ab)′2, Fab and Fv fragments. A preferred antibody is mAb 7-4 (available as of the filing date of this application from the University of Guelph Business Development Office, Guelph, Ontario, Canada). The term “mAb 7-4” as used herein means a monoclonal antibody specific for the inner core oligosaccharide of LPS and isolated as previously reported (de Kievit and Lam, 1994). Antibodies to LPS may be prepared using techniques known in the art. For example, by using a peptide of LPS, polyclonal antisera or monoclonal antibodies can be made using standard methods. A manual, (e.g., a mouse, hamster, or rabbit) can be immunized with an inmmunogenic form of the LPS antigen which elicits an antibody response in the manual. Techniques for conferring immunogenicity on an antigen include conjugation to carriers or other techniques well known in the art. For example, antigen can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g., the hybridoma technique originally developed by Kohler and Milstein (Nature 256, 495-497 (1975)) as well as other techniques such as the human B-cell hybridoma technique (Kozbor et al., Immunol. Today 4, 72 (1983)), the EBV-hybridoma technique to produce human monoclonal antibodies (Cole et al. Monoclonal Antibodies in Cancer Therapy (1985) Allen R. Bliss, Inc., pages 77-96), and screening of combinatorial antibody libraries (Huse et al., Science 246, 1275 (1989)). Hybridoma cells can be screened immunochemically for production of antibodies specifically reactive with the peptide and the monoclonal antibodies can be isolated. Therefore, the invention also contemplates hybridoma cells secreting monoclonal antibodies with specificity for LPS as described herein.

Antibodies can be fragmented using conventional techniques and the fragments screened for utility in the same manner as described above. For example, F(ab′)2 fragments can be generated by treating antibody with pepsin. The resulting F(ab′)2 fragment can be treated to reduce disulfide bridges to produce Fab′ fragments.

Chimeric antibody derivatives, i.e., antibody molecules that combine a variable region and a constant region are also contemplated within the scope of the invention. Conventional methods may be used to make chimeric antibodies containing the immunoglobulin variable region which recognizes the gene product of LPS antigens of the invention (See, for example, Morrison et al., Proc. Natl Acad. Sci. U.S.A. 81,6851 (1985); Takeda et al., Nature 314, 452 (1985), Cabilly et al., U.S. Pat. No. 4,816,567; Boss et al., U.S. Pat. No. 4,816,397; Tanaguchi et al., European Patent Publication EP171496; European Patent Publication 0173494, United Kingdom patent GB 2177096B). Chimeric antibodies are often less immunogenic than the corresponding non-chimeric antibody.

Specific antibodies, or antibody fragments, such as, but not limited to, single-chain F_{V} monoclonal antibodies reactive against LPS proteins may also be generated by screening expression libraries encoding immunoglobulin genes, or portions thereof, expressed in bacteria with LPS antigens. For example, complete F_{ab} fragments, V_{H} regions and F_{V} regions can be expressed in bacteria using phage expression libraries (See for example Ward et al., Nature 341, 544-546: (1989); Huse et al., Science 246, 1275-1281 (1989); and McCafferty et al. Nature 348, 552-554 (1990)). Alternatively, a SCID-hu mouse, for example the model developed by Genpharm, can be used to produce antibodies or fragments thereof.

In a preferred embodiment, antibodies are prepared as follows. Immunogen preparation. Immunogen for core-lipid A-specific hybridoma production was prepared according to the method of Bogard et al. (Bogard, W. C., D. L. Dunn, K. Abernethy, C. Kilgarriff, and P. C. Kung. 1987. Isolation and characterization of murine monoclonal antibodies specific for gram-negative bacterial lipopolysaccharide; association of cross-genus reactivity with lipid A specificity. Infect. Immun. 55:899-904), with minor modifications. Briefly, cells of a rough P. aeruginosa strain, AK1401, were suspended at a concentration of 5×10⁹ cells per ml in 1% (vol/vol) acetic acid and heated at 100° C. for 1 h. The cells were then washed with distilled water and lyophilized. Core fractions of LPS (i.e., LPS devoid of long-chain A-band or B-band polysaccahride) from strain AK1401 were prepared by extracting the LPS with phenol-hot water and then performing gel filtration fractionation with a Sephadex G-50 column (Pharmacia, Uppsala, Sweden).

Five milligrams of this core LPS was then suspended in 5 ml of 0.5% (wt/vol) triethylamide (Sigma, St. Louis, Mo.), after which 5 mg of the acid-treated bacteria was added. The mixture was stirred slowly for 30 min at room temperature and dried in vacuo with a Speed Vac centrifuge (Savant Instrument Inc., Hicksville, N.Y.).

Core-specific MAb production. Core LPS-coated cells were used to immunize BaLB/c mice intraperitoneally. A dose of 50 μl of core LPS-coated cell suspension per injection was used in a 1:1 mixture with Freud's incomplete adjuvant (Difco). Initially, the animals were immunized on days 0, 4, 9, 14, and 28. The injections were kept up once every 2 weeks unitl day 56. To test for a positive response against core LPS bands, Western immunoblots of sera from test bleeds were preformed. Upon detection of a positive reaction to bands in the core region, the animals were immunized once more and euthanized 3 days later to extract splenocytes for fusion with the myeloma cell line NS1. The fusion protocol and isolation of hubridoma clones were precisely as described previously by Lam et al. (Lam, J. S., L. A. MacDonald, M. Y. C. Lam, L. G. M. Duchesne, and G. G. Southam. 1987. Production and characterization of monoclonal antibodies against serotype strains of Pseudomonas aeruginosa. Infect. Immun. 55:1051-1057.) Hybridoma cell lines were screened for the production of anti-LPS antibodies by enzyme-linked immunosorbent assay (ELISA) and LPS purified from mutant strains of P. aeruginosa, including O5-derived mutants AK14L1 (core-pluspone O repeat), AK44 (completecore), AK1012, AK43, and 21-1 (core deficient) and O6-derived mutants A28 (complete core), R5 and H4 (core deficient). From the 340 hybridoma clones that were screened by ELISA, 14 were selected on the basis of therr reactivities to be aforementioned LPS antigens. These 14 hybridomas were cloned at least twice by limiting dilution and then further characterized. Purified LPS from the 20 P. aeruginosa sero-types, 9 other Pseudomonas species, Klebsiella pneumoniae, E. coli, and Ra-to-Re mutants of Salmonella were used as antigens in both ELISAs and Western blots (immunoblots) for further characterization of these MAbs. Clone 7-4 was identified as highly cross-reactive with LPS in immunoassays from all 20 standard serotypes of P. aeruginosa and other Pseudomonas species, including P. acidoviorans, P. chloraphis, P. syringae, P. putida, P. aureofaciens and P. stutzeri.

Recombinant Production of Antibodies

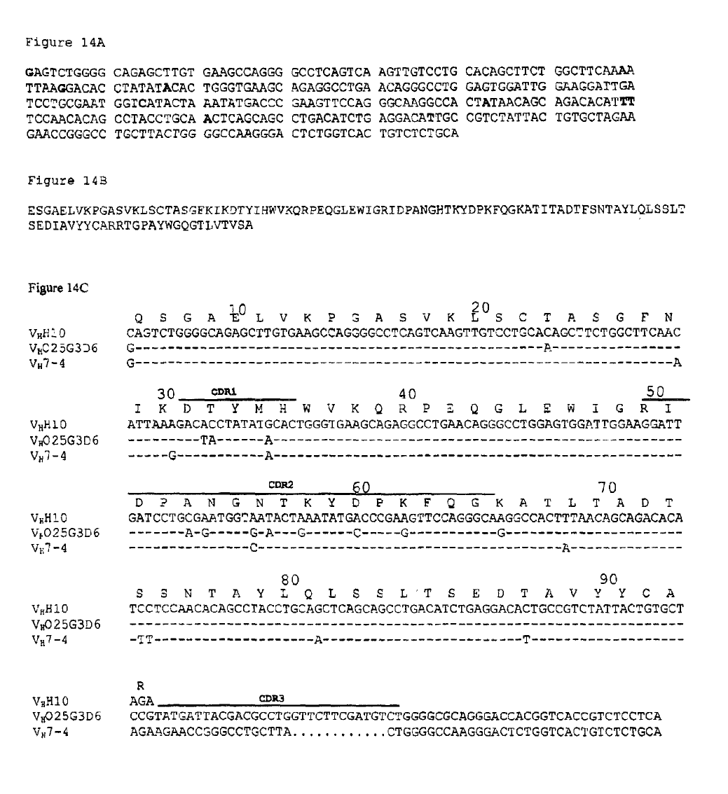

Antibodies, fragments and chimeric antibody derivatives suitable for use in the invention, as described in more detail above, may also be made recombinantly with an isolated nucleic acid encoding the 7-4 antibody or a fragment or variant of the nucleic acid. For example, the fragment may encode the variable heavy chain V region of 7-4 (FIG. 14), for example, the cloned VH fragment of 7-4 may be used to produce a recombinant antibody (Emara et al. J. Endotox. Res.). The heavy chain shown in FIG. 14 is preferably connected to a light chain to make a functional antibody.

Any of the recombinant 7-4F(ab) antibodies in the Emara et al. papers in J. Endotox. Res. (2:53-61, 1995) labeled as 7-4.r10.8, 7-4.r1.1, 7-4.r1.6, 7-4.r.1.7 etc. (also including recombinants designated 7-4.r1-7-4.r6 and 7-4.r7-4.r20) will work in the assays in place of mAb7-4 (the aforementioned antibodies are available as of the filing date of this application from the University of Guelph Business Development Office, Guelph, Ontario, Canada). 7-4.r10.8 could be used in immunofluorescence microscopy to light up P. aeruginosa bacterial cells. The invention includes a nucleic acid encoding a fusion protein that binds LPS, wherein the fusion protein comprises all or part of the polypeptide shown in FIG. 14 and a second polypeptide. The second polypeptide may be produced from other sequences for example, the VH-H10 nucleic acid, because mAb 7-4 is about 95% identical to the germline VH-H10 gene.

The term “isolated” refers to a nucleic acid substantially free of cellular material or culture medium when produced by recombinant DNA techniques, or chemical precursors, or other chemicals when chemically synthesized. The term “nucleic acid” is intended to include DNA and RNA and can be either double stranded or single stranded.

Since the hybridoma has been cloned messenger RNA coding for the heavy and light chain can be isolated employing standard techniques of RNA isolation and using oligo-dT cellulose chromatography to segregate the poly-A mRNA. A cDNA library is prepared from the mixture of RNA using a suitable primer. The primer is preferably a nucleic acid sequence which is characteristic of the desired cDNA. It the sequence of the antibody is known then the primer may be hypothesized based on the known amino acid sequence. cDNA must be used so that the DNA to be subsequently introduced into the selected host system is free from introns.

The cDNA sequences encoding individual light and heavy chains may be used to prepare a recombinant antibody, assembled by combining select light and heavy chain variable domains and available light and heavy chain constant domain sequences, respectively. Variable domains with specific binding properties may be isolated from screening populations of such sequences, usually in the form of a single-chain Fv phage display library. If the sequence of a variable domain is known for either the heavy or light chain, PCR primers can be readily designed to amplify the variable region which can subsequently be fused to the a human constant region for the appropriate heavy or light chain. If the sequence is not known, degenerate primers to immunoglobulin gene variable regions have been designed (see for example Wang et al. (2000) J. Immunological Methods 233: 167-177) for Reverse Transcription Polymerase Chain Reaction.

The nucleic acid sequences encoding the heavy and light antibody chains may be altered to improve expression levels for example by optimizing the nucleic acids sequence in accordance with the preferred codon usage for the particular cell type which is selected for expression of the heavy and light antibody chains.

The nucleic acid may be a variant such as a nucleic acid sequence that has substantial sequence identity to the nucleic acid sequence of FIG. 14 or an analog of a sequence in FIG. 14.

The term “sequence that has substantial sequence identity” means those nucleic acid sequences which have slight or inconsequential sequence variations from the sequence FIG. 14, i.e., the sequences function in substantially the same manner and can be used to produce an antibody that binds LPS. The variations may be attributable to local mutations or structural modifications. Nucleic acid sequences having substantial homology include nucleic acid sequences having at least 65%, more preferably at least 85%, and most preferably 90-95% identity with the nucleic acid sequence as shown in FIG. 14. Identity may be determined by reference the BLAST version 2.1 program advanced search (parameters as above). BLAST is a series of programs that are available online at http://www.ncbi.nlm.nih.gov/BLAST. The advanced blast search (http://www.ncbi.nlm.nih.gov/blast/blast.cgi?Jform=1) is set to default parameters. (ie Matrix BLOSUM62; Gap existence cost 11; Per residue gap cost 1; Lambda ratio 0.85 default).

References to BLAST searches are: Altschul, S. F., Gish, W., Miller, W., Myers, E. W. & Lipman, D. J. (1990) “Basic local alignment search tool.” J. Mol. Biol. 215:403_410; Gish, W. & States, D. J. (1993) “Identification of protein coding regions by database similarity search.” Nature Genet. 3:266_272; Madden, T. L., Tatusov, R. L. & Zhang, J. (1996) “Applications of network BLAST server” Meth. Enzymol. 266:131_141; Altschul, S. F., Madden, T. L., Schaffer, A. A., Zhang, J., Zhang, Z., Miller, W. & Lipman, D. J. (1997) “Gapped BLAST and PSI_BLAST: a new generation of protein database search programs.” Nucleic Acids Res. 25:3389_3402; Zhang, J. & Madden, T. L. (1997) “PowerBLAST: A new network BLAST application for interactive or automated sequence analysis and annotation.” Genome Res. 7:649_656.

The term “a nucleic acid sequence which is an analog” means a nucleic acid sequence which has been modified as compared to the sequence FIG. 14 wherein the modification does not alter the utility of the sequence as described herein. The modified sequence or analog may have improved properties over the sequence shown in FIG. 14. One example of a modification to prepare an analog is to replace one of the naturally occurring bases (i.e. adenine, guanine, cytosine or thymidine) of the sequence with a modified base such as such as xanthine or hypoxanthine.

Isolated and purified nucleic acid molecules having sequences which differ from the nucleic acid sequence of the invention due to degeneracy in the genetic code are also within the scope of the invention. Such nucleic acids encode functionally equivalent proteins but differ in sequence from the above mentioned sequences due to degeneracy in the genetic code.

A nucleic acid molecule of the invention may also be chemically synthesized using standard techniques. Various methods of chemically synthesizing polydeoxynucleotides are known, including solid-phase synthesis which, like peptide synthesis, has been fully automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071).

The antibodies described above are useful for detection of bacteria having LPS by contacting the antibody with a test sample (such as a sample from a human or other mammal) that may contain bacteria (the sample may also contain an extract of the bacteria). Binding of the antibody to bacterial LPS indicates that the bacteria is present in the test sample.

### Assays

As previously stated, an enzyme-linked immunosorbent assay (ELISA) has been developed for the determination of the enzyme activity of WaaP to phosphorylate the inner core oligosaccharide of LPS (Zhao et al. 2002). HF-LPS, the dephosphorylated LPS obtained by hydrofluoric acid (HF) treatment, was generated, characterized and used as the substrate in the enzyme assay. A monoclonal antibody 7-4, previously reported to be specific for the inner core oligosaccharide (de Kievit and Lam, 1994), was found to specifically recognize the phosphate group(s) on LPS and therefore, was used as the primary antibody in the ELISA.

The present invention therefore relates to a method of assaying for modulators of an enzyme involved in the phosphorylation of the inner core oligosaccharide of LPS, comprising the steps of:

(a) incubating a test sample comprising (i) the enzyme, (ii) a substance suspected of being a modulator of the enzyme; and (iii) substrates comprising dephosphorylated LPS and a source of phosphate;

(b) preferably stopping the reaction;

(c) adding at least one antibody comprising an antibody that binds to phosphorylated LPS while not binding to dephosphorylated LPS; and

(d) quantifying the amount of phosphorylated LPS in the test sample by measuring the binding of the at least one antibody to phosphorylated LPS, wherein a change in the amount of phosphorylated LPS in the test sample compared to an amount of phosphorylated LPS in a control sample (that does not contain the substance suspected of being a modulator) indicates that the substance is a modulator.

Preferably, the assay is used to screen for inhibitors of an enzyme involved in the phosphorylation of the inner core oligosaccharide of LPS. Therefore the present invention further relates to a method of assaying for inhibitors of an enzyme involved in the phosphorylation of the inner core oligosaccharide of LPS comprising the steps of:

(a) incubating a test sample comprising (i) the enzyme, (ii) a substance suspected of being an inhibitor of the enzyme; and (iii) substrates comprising dephosphorylated LPS and a source of phosphate;

(b) preferably stopping the reaction;

(c) adding at least one antibody comprising an antibody that binds to phosphorylated LPS while not binding to dephosphorylated LPS; and

(d) quantifying the amount of phosphorylated LPS in the test sample by measuring the binding of the at least one antibody to phosphorylated LPS, wherein a decrease in the amount of phosphorylated LPS in the test sample compared to an amount of phosphorylated LPS in a control sample (that does not contain the substance suspected of being an inhibitor) indicates that the substance is an inhibitor.

The enzyme assayed using the method of the invention may be any kinase involved in the phosphorylation of the inner core oligosaccharide of LPS, preferably in Gram negative bacteria. Preferably, the kinase is a heptose kinase involved in the phosphorylation of a heptose residue in the inner core oligosaccharide of LPS, preferably in Gram negative bacteria. Most preferably, the enzyme is WaaP, or a homologue of WaaP. The term “homolog” as used herein means that a particular subject sequence or molecule, for example, a mutant sequence, varies from a reference sequence by one or more substitutions, deletions, or additions, the net effect of which does not result in an adverse functional dissimilarity between reference and subject sequences. For purposes of the present invention, amino acid sequences having greater than 60 percent and more preferably greater than 90 percent sequence identity, equivalent biological activity, and equivalent expression characteristics are considered substantially homologous and are included within the scope of proteins defined by the terms “enzyme involved in the phosphorylation of the inner core oligosaccharide of LPS” and/or “homologs of WaaP”. Sequence identity may be determined using BLAST. Amino acid sequences having greater than 40 percent similarity are considered substantially similar. For purposes of determining sequence identity, truncation or internal deletions of the reference sequence should be disregarded, as should subsequent modifications of the molecule, e.g., glycosylation. Sequences having lesser degrees of homology and comparable bioactivity are considered equivalents. Specific examples of kinases involved in the phosphorylation of the inner core oligosaccharide in Gram negative bacteria include E. coli WaaP (WaaP_{Ec}) (Yelthon, 2001), P. aeruginosa WaaP (WaaP_{Pa}) (Walsh 2000) and S. typhimurium WaaP (Helander et al. 1989). WaaP of E. coli, Salmonella typhimurium and P. aeruginosa are functionally interchangeable with each other, as shown in Walsh et al. (2000): WaaPEc and WaaPSt (Salmonella typhimurium) are 83% identical and 93% similar to each other;

WaaPPa and WaaPEc are 51% identical and 69% similar to each other; and WaaPPa and WaaPSt are 52% identical and 72% similar to each otherPreferably the enzyme is WaaPPa. It will be readily understood by those skilled in the art, that the enzyme can be expressed in a form which may be rapidly purified (such as a His-tagged construct with 6 histidine residues at its C- or N-terminus) using any one of a number of chromatographic methods known to those skilled, such as, for example, metal chelate chromatography in open column or High Performance Liquid Chromatography (HPLC) formats.

For developing an assay to determine the phosphorylation activity of an enzyme involved in the phosphorylation of the inner core saccharide of LPS, for example WaaP, LPS without phosphate was generated to serve as the substrate. HF treatment is the standard dephosphorylation method used in structure analysis of core oligosaccharide in LPS (Dasgupta, 1994; Kondo, 1992; Katzenellenbogen, 1998; Toukach, 1996). In the results reported herein, HF also degraded the long chain LPS to some extent. However, there was no evidence that HF treatment affected the structure of the core oligasaccharide. The term “HF-LPS” as used herein means a dephosphorylated LPS obtained by hydrofluoric acid (HF) treatment of LPS. An advantage of using HF-LPS is the fact that this modified form of LPS contains much more shorter-chain than wild type PAO1-LPS. The term “PAO1-LPS” as used herein means the wild type LPS from P. aeruginosa strain PAO1, serotype O5. The much shorter O-antigen chain may provide less steric hindrance for the enzyme to reach the sugar moiety, for example heptose, during the enzymatic phosphorylation. A purified LPS containing lipid A attached to a phosphate-deficient core oligosaccharide as the acceptor molecule may also be extracted from a prototype E. coli waaP mutant strain (which has a phosphate-deficient core oligosaccharide) such as, for example strain CWG296 (Yethon et al., 1998).

The method of the invention therefore involves assaying for inhibitors of an enzyme involved in the phosphorylation of the inner core oligosaccharide of LPS, using as the substrates, dephosphorylated LPS and a source of phosphate. The source of phosphate may be any such source, preferably adenosine triphosphate (ATP) or guanosine triphosphate (GTP). The reaction mixture may also comprise other reagents such as magnesium chloride (MgCl₂, which forms a salt bridge that is involved in proton transfer in enzymatic reactions) and dithiothreotol (DTT, a reducing agent that serves to stabilize ATP). Other salt containing cations, for instance, CaCl₂ and MnCl₂, may also be used. The reaction mixture is preferably buffered to a pH of approximately 7-9, preferably 7.2-7.9, using, for example a Tris-HCl buffer.

In an embodiment of the invention, the assay is performed by preparing a solution comprising dephosphorylated LPS, ATP, MgCl₂ and DTT in a buffer solution at approximately pH 7.8. The reaction is initiated by the addition of the kinase enzyme, for example, WaaP. The reaction is incubated, preferably at about 37° C., for the appropriate amount of time, for example about 10 minutes to about 1 hour. The reaction may be stopped, or quenched, for example, by the addition of a chloroform/ethanol (1/10) solution.

Quantitation of the extent of the reaction is preferably done using the enzyme-linked immunosorbent assay (ELISA). The identification of the phosphate of LPS as the epitope of mAb 7-4 in this study allowed the development of an ELISA as a non-radio-labeling assay for inner core LPS kinases. It is understood that the method of the invention would work using any antibody that binds to phosphorylated LPS while not binding dephosphorylated LPS. A person having skill in the art would be able to obtain an antibody that binds to phosphorylated LPS while not binding to dephosphorylated LPS using standard procedures, for e.g. as described in de Kievit and Lam, 1994. Preferably this antibody is mAb 7-4. The isolation and characterization of mAb 7-4 has been described (de Kievit and Lam, 1994). Preferably, the antibody mixture used in the ELISA comprises both the primary antibody mAb 7-4 and secondary antibody, for example alkaline phosphatase conjugated-goat anti-mouse F(ab′)₂ to allow simultaneous incubation of the antibodies. The use of this strategy further simplifies and shortens the ELISA procedure. The ELISA may be performed using standard protocols, for example those described in Bantroch et al. (1994). The ELISA may be developed using a calorimetric method (Bantroch, 1994) or a chemiluminescence method. Preferably a chemiluminescence method is used.

The methods of the invention can be carried out in nearly any reaction vessel or receptacle. Examples of suitable receptacles include 96-well plates, 384-well plates, test tubes, centrifuge tubes, and microcentrifuge tubes. The methods can also be carried out on surfaces such as on metal, glass, or polymeric chips, membrane surfaces, the surface of a matrix-assisted laser-desorption ionization mass spectrometry (MALDI-MS) plate, on a resin, and on a glass, metal, ceramic, paper, or polymer surface.

As appropriate in identifying substances which modulate the activity of an enzyme involved in the phosphorylation of the inner core oligosaccharide of LPS, the enzyme, or the modulating substrate, used in the method of the invention may be insolubilized. For example, WaaP (or its homologues) or a substrate of WaaP (or its homologues) may be bound to a suitable carrier. Examples of suitable carriers are agarose, cellulose, dextran, Sephadex, Sepharose, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, beads, disc, sphere etc. The insolubilized enzyme or substance may be prepared by reacting the material with a suitable insoluble carrier using known chemical or physical methods, for example, cyanogen bromide coupling. The use of carriers for binding substrates and/or proteins in biological assays, in particular in assays for identifying novel antibiotics, is described in U.S. Pat. No. 6,043,045, the contents of which are incorporated herein by reference.

The data obtained using the method of the invention may be converted, for example, to a Kᵢ, EC₅₀ and/or IC₅₀ value for the test substance using standard protocols known to those skilled in the art. In a further aspect of the invention, data obtained in the instant assays are recorded via a tangible medium, e.g., computer storage or hard copy versions. The data can be automatically input and stored by standard analog/digital (A/D) instrumentation that is commercially available. Also, the data can be recalled and reported or displayed as desired for best presenting the instant correlations of data. Accordingly, instrumentation and software suitable for use with the present methods are contemplated as within the scope of the present invention.

### Uses

Substances which affect activity of an enzyme involved in the phosphorylation of the inner core oligosaccharide of LPS can be identified based on their ability to modulate the activity of the enzyme. Therefore, the invention provides methods, as described above, for identifying substances which are capable of modulating the activity of an enzyme involved in the phosphorylation of the inner core oligosaccharide of LPS, preferably WaaP. In particular, the methods may be used to identify substances which are capable of inhibiting the activity of an enzyme involved in the phosphorylation of the inner core oligosaccharide of LPS, in particular WaaP. Substances that inhibit the activity of an enzyme involved in the phosphorylation of the inner core oligosaccharide of LPS, in particular WaaP, may be useful as antibiotics.

The methods of the invention may be used to screen a wide variety of compounds or compound libraries for activity as modulators and/or inhibitors of enzymes involved in the phosphorylation of the inner core oligosaccharide of LPS. A library of potential modulators and/or inhibitors can be a natural compound library, a synthetic combinatorial library (e.g., a combinatorial chemical library), a cellular extract, a bodily fluid (e.g., urine, blood, tears, sweat, or saliva), or other mixture of synthetic or natural products (e.g., a library of small molecules or a fermentation mixture).

A library of potential inhibitors can include, for example, amino acids, oligopeptides, polypeptides, proteins, or fragments of peptides or proteins; nucleic acids (e.g., antisense; DNA; RNA; or peptide nucleic acids, PNA); aptamers; or carbohydrates or polysaccharides. Each member of the library can be singular or can be a part of a mixture (e.g., a compressed library). The library can contain purified compounds or can be “dirty” (i.e., containing a significant quantity of impurities).

Commercially available libraries (e.g., from Affymetrix, ArQule, Neose Technologies, Sarco, Ciddco, Oxford Asymmetry, Maybridge, Aldrich, Panlabs, Pharmacopoeia, Sigma, or Tripose) can also be used with the methods of the invention.

Yet another aspect of the present invention provides a method of conducting a target discovery business comprising:

(a) providing one or more assay systems for identifying agents by their ability to modulate an enzyme involved in the phosphorylation of the inner core oligosaccharide of LPS, said assay systems using a method of the invention;

(b) (optionally) conducting therapeutic profiling of agents identified in step (a) for efficacy and toxicity in animals; and

(c) licensing, to a third party, the rights for further drug development and/or sales or agents identified in step (a), or analogs thereof.

By assay systems, it is meant, the equipment, reagents and methods involved in conducting a screen of compounds for the ability to modulate an enzyme involved in the phosphorylation of the inner core oligosaccharide of LPS using the method of the invention.

### Kits

The reagents suitable for carrying out the methods of the invention may be packaged into convenient kits providing the necessary materials, packaged into suitable containers. For example the reagents may include reagents for performing the enzyme reaction, such as an aliquot of dephosphorylated LPS, for example, HF-LPS, an aliquot of ATP, an aliquot of the kinase of interest (for example WaaP or its homolgue) and buffer, and reagents for performing the ELISA, for example an aliquot of an antibody that binds to phosphorylated LPS while not binding to dephosphorylated LPS, preferably mAb 7-4, an aliquot of a secondary antibody and reagents specific for either colorimetric or chemiluminescence analysis.

With particular regard to assay systems packaged in “kit” form, it is preferred that assay components be packaged in separate containers, with each container including a sufficient quantity of reagent for at least one assay to be conducted. A preferred kit is typically provided as an enclosure (package) comprising one or more containers for the within-described reagents.

The reagents as described herein may be provided in solution, as a liquid dispersion or as a substantially dry powder, e.g., in lyophilized form. Usually, the reagents are packaged under an inert atmosphere.

Printed instructions providing guidance in the use of the packaged reagent(s) may also be included, in various preferred embodiments. The term “instructions” or “instructions for use” typically includes a tangible expression describing the reagent concentration or at least one assay method parameter, such as the relative amounts of reagent and sample to be admixed, maintenance time periods for reagent/sample admixtures, temperature, buffer conditions, and the like.

The following non-limiting examples are illustrative of the present invention:

### EXAMPLES

### Materials and Methods

Amino acid alignment analysis of WaaP_{Pa} with WaaP_{Ec} and protein kinases—Amino acid of WaaP was aligned with the protein kinases in the subdomains stated in the nomenclature of Hanks (Hanks, 1991). The alignment of WaaP_{Pa} and WaaP_{Ec} was performed by Basic Local Alignment Searching Tool (BLAST) accomplished by using database Non-redundant GenBank CDS (Altschul, 1997).

Site-directed mutagenesis and in vivo complementation assay—waaP was amplified by polymerase chain reaction (PCR) from pCOREc1 (de Kievit and Lam, 1997) with the flanking up- and down-stream primers ^{5′}ATAATAGGATCCATGAGGCTGGTGCTGG^{3′} (SEQ ID NO: 3) and ^{5′}TATATTAAGCTTCAGAGCAGGTCTCCG^{3′} (SEQ ID NO: 4) containing Baam HI and Hind III respectively. The PCR product was cloned into the complementation vector pUCP26 (West, 1994) as a positive control for complementation assay. Mutations of waaP were introduced by the method of “overlapping extension” as described by Horton (Horton, 1993) using PCR with the flanking primers as well as the primers shown below. K69A mutation was introduced with the primer (only up-strand was described here) ^{5′}GCTCACCGCCGCGCTCCCGGTG^{3′} (SEQ ID NO: 5); K69R with ^{5′}GCTCACCGCCAGGCTCCCGGTGCTCGGC^{3′} (SEQ ID NO: 6); D163A with ^{5′}CAACCATCGCGCCTGCTACATCTGTC^{3′} (SEQ ID NO: 7); and D163E with ^{5′}CAACCATCGCGAGTGCTACATCTGTC^{3′} (SEQ ID NO: 8). Underlined nucleotides indicated the mutations. The PCR products were then cloned into pUCP26 at Bam HI and Hind III sites respectively, and transformed into E. coli F470 7waaP- (Yethon 1998). Constructs of the mutations of waaP were sequenced to confirm the mutation. In vivo complementation was tested for the minimum inhibition concentration (MIC) of SDS and novobiocin, respectively according to Walsh, et al. 2000. E. coli F470 7waaP- was used as the negative control.

Cloning of waaP into an expression vector—waaP was amplified by PCR using pCOREc1 as the template, which contained the core gene cluster of P. aeruginosa (Walsh, 2000). The forward- and reverse-primers were ^{5′}TATATATCATATGAGGCTGGTGCTGG^{3′} (SEQ ID NO: 9) and ^{5′}TATATAAGCTTAGAGAGCAGGTCTCCG^{3′} (SEQ ID NO: 10 ) Containing Nde I and Hind III restriction endonuclease sites, respectively. The down-stream primer also contained the mutation (underlined) to change the stop codon TGA to TCT for waaP. This PCR product was cloned into pET30a (+) expression vector (Novagen, Madison, Wis.) at Nde I and Hind III sites to be in frame with the 6×His tag at the C-terminus of the protein. The construct was then introduced into E. coli JM109 by CaCl₂ transformation (Huff, 1990). Transformants were selected on Luria agar (Fisher Scientific Co, Hanover Park, Ill.) containing 30 mg·l⁻¹ kanamycin. Both strands of DNA were sequenced to confirm the sequence of the cloned waaP. The resultant construct waaPHisC was overexpressed in E. coli BL21(DE3)pLysS (Novagen). All the chemicals used in this paper were from Sigma (St. Louis, Mo.) unless stated.

Overexpression of the plasmid encoded WaaPHisC—Terrific broth (TB, Sambrook, 1989) supplemented with 3 mg·l⁻¹ kanamycin and 3.4 mg·l⁻¹ chloramphenicol was used for the overexpression of WaaPHisC. The cells were first cultivated with shaking at 37° C. to 0.6 at A_{600 nm}. The overexpression of recombinant protein was induced with 1 mM isopropyl-β-O-thiogalactopyranoside (IPTG) for 3.5 h. Cells were harvested by centrifugation at 5,000×g and pellets were frozen at −20° C. pET30a/E. coli BL21(DE3)plysS (Novagen) was used as the control for comparison with the overexpression of WaaPHisC.

Purification of WaaPHisC—Two grams of frozen cell pellet was suspended in 20 ml Tris-buffer (20 mM Tris-HCl, 0.5 M NaCl, pH 8.0) containing 5 mM imidazole and 10 mM β-mercaptoethanol. A protease inhibitor cocktail which contains 4-(2-aminoethyl)benzenesulfonyl fluoride, bestatin, pepstatin A, trans-epoxysuccinyl-L-leucylamido(4-guanidino)butane (E-64), and N-(α-rhamnopyranosyloxyhydroxyphosphiny)-Leu-Trp(phosphoramidon) was added. Cells were broken by sonication on ice for 2 min (Ultrasonic Processor SL 2020, MANDEL Scientific Company Ltd., Guelph, ON) followed by centrifugation at 10,000×g at 4 ° C. for 20 min. The supernatant containing the soluble protein of WaaPHisC was mixed with 3 ml of Cobalt-based immobilized metal affinity chromatography (IMAC) resin (TALON metal affinity resin with a capacity of 12 mg polyhistidine-tagged protein per ml of resin, CLONTECH Laboratories, Inc, Palo Alto, Calif.) and incubated at 4° C. for 1 h with gentle shaking. Then the mixture was loaded onto a 1.6 cm diameter column, and washed with 20 bed volumes of 5 mM imidazole/Tris-buffer. After the column was further washed with 10 bed volumes of 20 mM imidazole/Tris-buffer and the protein of WaaPHisC was eluted with 1 M imidazole/Tris-buffer.

The eluted protein was dialyzed extensively at 4° C. against 20 mM Tris-HCl, pH 8 using the dialysis tubing with 3,000 MWCO (Spectrum Laboratories, Inc. Rancho Dominguez, Calif.), and concentrated with polyethylene glycol 8000.

Protein assay—Protein concentration was determined by the BCA method (Smith, 1985) following the procedure described by the manufacturer (Pierce, Rockford, Ill.), and bovine serum albumin (BSA) was used as the standard.

SDS-polyacrylmide gel electrophoresis (SDS-PAGE) and Western immunoblotting—Purified protein was analyzed by a standard SDS-polyacrylamide gel electrophoresis method using 12.5% resolving gel (Laemmli, 1970) and stained with Coommassie blue R250. SeeBlue™ Pre-Stained standards (NOVEX, Scarborough, ON) were used as the molecular weight marker. Western immunoblotting following SDS-PAGE was performed using nitrocellulose membrane according to Burnette (Burnette, 1981) using Penta-His® Antibody (Qiagen, Mississauga, ON) diluted to 1:1,000 in 3% BSA/TBS (according to the Manufacturer's Instruction, Qiagen) as the primary antibody. Alkaline phosphatase conjugated-goat antimouse F(ab′)₂ Jackson ImmunoResearch Laboratory, Inc., Mississauga, ON), diluted at 1:2,000 in 3% BSA/TBS, was used as the secondary antibody. For the detection of phosphotyrosine in Western immunoblotting, the primary antibody used for detection of phosphotyrosine was Phosphotyrosine—PY20 Antibody (1:3,000 diluted in 3% BSA) (Transduction Laboratories, Lexington, Ky.) on a microporous polyvinylidene difluoride (PVDF) membrane (Roche Diagnostics Co, Indianapolis, Ind.).

SDS-PAGE gel of LPS was stained by using the rapid silver staining method of Formsgaard (Formsgaard, 1990). Different primary monoclonal antibodies described previously by our group (Bantroch, 1994) including mAb 7-4 (inner core specific), MF15-4 (B-band specific), N1F10 (A-band specific), 5c101 (outer core specific) and 18-19 (core+one antigen unit specific) were used in the Western immunoblotting analysis of LPS on the nitrocellulose membrane, and they were supernatant of cell cultures. PBS-0.1% Tween 20 was used as the washing buffer except that no Tween 20 was used for the Western immunoblotting using mAb N1F10 as the primary antibody.

Matrix-assisted laser desorption/ionization time-of-flight (MALDI-TOF) mass spectrometry—MALDI-TOF mass spectrometry of WaaPHisC was performed locally at the University of Guelph with a Bruker-Relex (Bruker-Franzen Analytik, Bremen, Germany) in reflector configuration at an acceleration voltage of 20 kV and delayed ion extraction. The sample was dissolved in 0.2% fluoroacetic acid and 50% acetonitrile. An aliquot of 0.5 μl was deposited on a metallic sample holder and analyzed immediately after drying in air. Mass spectrum was recorded in the negative ion mode. Cytochrome c and carbonic anhydrase were used to calibrate the molecular mass.

Peptide mapping on proteolytic digested WaaPHisC—IMAC purified WaaP was digested with proteases including trypsin, chymotrypsin and endoproteinase ArgC (sequence grade, Roche Diagnostics Co, Indianapolis, IN), separately, at 10 μg protein/μg protease in 20 μl solution by incubating at 30° C. for 24 h. After mixed with trifluoacetic acid (TFA) and GdnHCl to the final concentrations of 1% and 1 M respectively, the peptides were loaded onto a Zip-TipC₁₈ pipette tip for purification. The purified peptides were eluted with 5 μl 50% acetonitrile in 0.1% TFA, and 0.5 μl was used for MALDI-TOF analysis. The phosphorylated tyrosin residues in WaaP were identified by comparing the actual mass of the individual peptide (from MALDI-TOF analysis) with the predicted mass of the corresponding peptide that were obtained from the on-line analysis tool using “Peptide Mass” program in www.expasy.ch.

Preparation of PAO1-LPS—P. aeruginosa PAO1 cells were cultivated in 300 ml of LB overnight and harvested by centrifugation at 6,000×g for 10 min. After the cells were washed 2 times with PBS (phosphate buffered saline, containing 0.8% NaCl, 0.02% KH₂PO₄, 0.29% Na₂HPO₄, 0.05% KCl, pH 7.4), LPS was extracted by the standard hot water-phenol method of Westphal (Westphal, 1965), and then it was lyophilized until use.

Preparation of HF-LPS—The wild-type LPS was dephosphorylated with 48% hydrofluoric acid (HF) at 4° C. for 48 h, dialyzed extensively against H₂O, and the HF-LPS was recovered by lyophilyzation (Kondo, 1992).

Phosphate analysis—An aliquot of 25 μl containing 0.25 mg LPS was applied onto the nitrocellulose membrane (0.22 μm porous size and 25 mm diameter) on the manifold (Millipore Corporation, Bedford, Mass.). The membrane was washed with 50 ml H₂O by adding slowly to avoid accumulation of H₂O on the surface of the membrane. Then the whole membrane was removed from the manifold, folded, and put on the bottom of a 1 cm diameter borostlicate glass test tube. The membrane was then wet with 200 μl 10% Mg(NO₃)₂.H₂O in ethanol, evaporated and ashed on the burner until brown fumes disappeared. After the tube cooled down, 0.3 ml 1 N HCl was added, and then the tube was capped with marble and boiled for 15 min in boiling water bath. The inorganic phosphate was determined according to Ames et al. (1960) (Ames, 1960). Briefly, 0.7 ml ascorbic-molybdate mixture containing 1 part of 10% ascorbic acid and 6 parts of 0.42% ammonium molybdate.4H₂O in 1 N H₂SO₄ was added (freshly prepared daily). After incubation at 45° C. for 20 min, the mixture was centrifuged at 12,000×g for 10 min and A_{820 nm} of the supernatant was measured. The same procedure was performed with 25 μl H₂O on the nitrocellulose membrane as the negative control. K₂HPO₄ of 0-40 nmol was used as the standard.

Determination of critical aggregation concentration of LPS—LPS solutions were prepared in 100 μl 20 mM Tris-HCl, 150 mM NaCl, pH 7.5. Serial two fold dilutions were prepared starting with 5 mg·ml¹ LPS in glass tubes. Then 5 μM of N-phenyl-1-naphthylamine (NPN), the fluorescence marker, was added to each tube, mixed and incubated for 30 min at room temperature. Before the incubation time was over, 50 μl of this solution was quickly transferred into a black 96-well microtiter plate (FluoroNunc Module with MaxiSorp surface, Fisher Scientific, Ottawa, ON). As the NPN molecule partitions into the hydrophobic compartment of the LPS aggregates, its emission peak shifted from about 475 to 425 nm. Fluorescence was measured with excitation wavelength at 350 nm and emission at 425 nm. The graph of the Relative Fluorescence as the function of Log₁₀ [LPS concentration (mg·ml⁻¹)]×10⁵ was analyzed and the CAC of LPS can be determined by the intercept of the increasing part of each curve where the aggregation of LPS started to form.

Enzyme-linked immunosorbent assay (ELISA) for WaaPHisC—ELISA was performed using the polystyrene, high binding, 96-well microtiter plate (transparent plate was used for the colorimetric reading and the opaque plate for the chemiluminescence reading) (Corning Incorporated, Corning, Buslinch, ON). Antigen coating was accomplished by adding 50 μl LPS or enzyme reaction mixture to each well and then mixing with equal amount of chloroform-ethanol (CH₃Cl/EtOH) (1:10, v/v)). The plate was incubated at room temperature in the fume hood (flow rate at 150 ft·min⁻¹) overnight to allow the evaporation of the solvent. The ELISA was performed according to Bantroch et al. (1994) (Bantroch, 1994) with the following modifications. The antibody mixture containing primary antibody mAb 7-4 (at 1:20 dilution) and secondary antibody alkaline phosphatase conjugated-goat anti-mouse F(ab′)₂ (at 1:2,000 dilution) (Jackson Immunoscientific, Richmond, VC ) in 1% skim milk/PBS was used and incubated at 37° C. for 2 h. For the colorimetric method (Bantroch, 1994), the ELISA was developed at 37° C. for 2 h and the optical density at 405 nm was determined on a microplate reader (Flow Laboratories, Mississauga, ON). For the chemiluminescence development, 100 μl of the chemiluminescence substrate CDP-Star® Ready-to-Use with Emerald-II™ (CDP*) (Applied Biosystems, Bedford, Mass.) with 1:5 (v/v) diluted in diethanolamine buffer (9.6% (v/v) and 0.01% (w/v) MgCl₂, pH 9.8) was added to each well. After incubation at room temperature for 20 min, the response of chemiluminescence was measured on a 1420-VICTOR² Multilabel Counter (Wallac, Montreal, QC) using chemiluminescence program.

Enzymatic reconstitution of HF-LPS by WaaPHisC—Phosphorylation of HF-LPS enzyme reaction was performed in a 96-well microtiter plate in 50 μl solution containing 100 ng HF-LPS, 20 mM MgCl₂, 50 mM dithiothreotol (DTT), 250 μM ATP in 20 mM Tris-HCl buffer, pH 7.8 and the reaction was started by the addition of 5 μg of enzyme (purified WaaPHisC in 20 mM Tris-HCl, pH 7.5). The reaction mixture was incubated at 37° C. for 30 min (15 min for kinetics experiments) and quenched by the addition of 50 μl chloroform/ethanol (1:10) solution. The plate was then left at room temperature in the fume hood overnight and subjected to ELISA the next morning. PAO1-LPS was used as the standard to quantify phosphorylated LPS.

### Results

### Example 1

### Characterization of WaaP

(a) Amino acid sequence alignment analysis

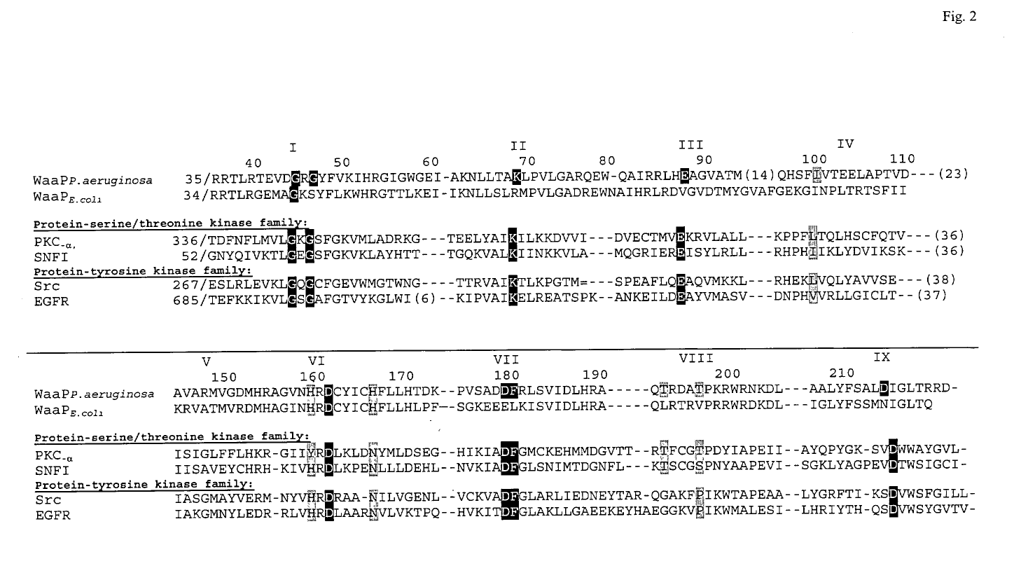

Genetic evidence to show that WaaP is a sugar (heptose) kinase has been previously provided (Walsh, 2000). To further investigate its kinase function and compare it with other kinases including protein kinases, alignment comparisons between the amino acid sequence of WaaP and those of a number of the well-characterized protein kinases from eukaryotes were performed (FIG. 2). Since WaaP_{Pa} and WaaP_{Ec} shared 52% identity on amino acid sequence, both WaaP amino acid sequences were also aligned and compared with the protein kinases. In FIG. 2, two members of protein kinases (PKC-alpha and SNF1) from serine/threonine kinase (PKC) family and two (Src and EGFR) from tyrosine kinase family (Src) family were selected, respectively, for the alignment analysis with the sequence of WaaP_{Pa}. The sequences of these protein kinases can be divided into twelve subdomains (I-XI) according to the nomenclature of Hanks (Hanks, 1988; 1991). Only subdomain I-IX is shown in FIG. 2. The results indicated that WaaP has significant identity on the conserved, functional residues of the protein kinases. Subdomain I is rich in glycine residues, and the G⁴⁵XG or G⁵⁵XGXG (X can be any amino acid) (Wierenga, 1983) is the signature of the nucleotide binding. K⁶⁹ in subdomain II is the well-characterized catalytic domain residue that is involved in the proton transfer in the phosphotransfer reaction (Kamps, 1986). In the central core of the catalytic domain VI through IX, the invariant residues D¹⁶³, D¹⁸¹ and F¹⁸² have been implicated in ATP binding and this is also the feature of some bacterial phosphotransferases that use ATP as the phosphate donor (Hanks, 1988). Furthermore, D¹⁶³ and D¹⁸¹ may interact with the phosphate groups of ATP through Mg²⁺salt bridge (Brener, 1987; Hanks, 1988; 1991; Madhusudan, 1994). These characteristics of WaaP suggested that in addition to the function as a sugar kinase, this enzyme might also be a protein kinase.

In contrast, the sequence of WaaP_{Ec} did not align well on all the functional motifs with the protein kinases. It did not contain the signature of the nucleotide binding site (GXG) in subdomain I, and therefore, the catalytic lysine in subdomain II, which is corresponding to K⁶⁹ in WaaP_{Pa}, is difficult to be localized as well as the glutamate in subdomain Ill. The only good alignment of WaaP_{Ec} is the catalytic domain HRD (corresponding to H¹⁶¹RD in WaaP_{Pa}) in subdomain VI. Then again, it did not show good alignment with D¹⁸¹F of WaaP_{Pa}. Thus, these results showed that WaaP_{Ec} does not contain the typical pattern of the characteristic functional motifs that are the characteristic of tyrosine kinases.

To validate the accuracy of the alignment comparisons in FIG. 2, site-directed mutagenesis of waaP_{Pa} was performed targeting on K⁶⁹ and D¹⁸¹, respectively. The effect of the site-directed mutation was evaluated by their ability to complement waaP-_{Ec}. It is noteworthy that the complementation assay was performed using waaP-_{Ec} since waaP mutation is lethal to P. aeruginosa. In Table I, the complementation of waaP-_{Ec} by wild type waaP_{Pa} increased the MICs of waaP-_{Ec} by 3 and 30 times to novobiocin and SDS, respectively. However, the MICs of all the mutants were at the same level as that of waaP-_{Ec}. This indicated that K⁶⁹ or D¹⁸¹ were essential residues for the function of waaP of P. aeruginosa. Therefore, these results proved the alignment of WaaP_{Pa} with the protein kinases shown in FIG. 2.

TABLE I
Minimum inhibition concentration (MIC) of novobiocin and
SDS for E. coli F470 waaP⁻ mutant
complemented with P. aeruginosa waaP gene and the
mutants of waaP_{Pa} generated by site-directed
mutagenesis.

| | MIC (μg.ml⁻¹) | |
|---|---|---|
| Strain | Novobiocin | SDS |
| F470 (wild type) | >200 | >50,000 |
| E. coli F470 (waaP⁻) | 50 | 400 |
| E. coli F470 (waaP⁻)/waaP_{Pa} | 150 | 12,500 |
| E. coli F470 (waaP⁻)/waaP_{Pa}(K69A)* | 50 | 400 |
| E. coli F470 (waaP⁻)/waaP_{Pa}(K69R)* | 50 | 400 |
| E. coli F470 (waaP⁻)/waaP_{Pa}(D163A)* | 25 | 400 |
| E. coli F470 (waaP⁻)/waaP_{Pa}(D163E)* | 50 | 200 |

*K69A designates that Lys 69 of WaaP_{Pa} was mutated to Ala and the same designation applies for the other site directed mutants in the table. The same convention was used for the designation of the next 3 recombinant strains with site-directed mutagenesis performed as K69R, D163A, and D163E etc.

(b) Purification of WaaPHisC

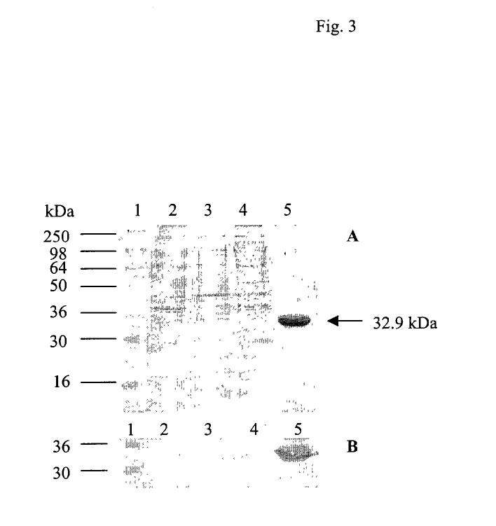

Results from SDS-PAGE and the corresponding Western immunoblotting with anti-Penta-His antibody showed that the 6×His tag was expressed as part of WaaP and a band with an apparent molecular mass of 33 KDa was observed. This is very close to the predicted molecular mass of 32.9 KDa (the mass without 6×His tag should be 31.3 KDa). The solubility assay performed on the recombinant WaaPHisC demonstrated that >90% of this protein was expressed in the soluble form (data not shown). The IMAC purification of WaaPHisC has been optimized and the yield obtained was 0.5 mg protein·l⁻¹ culture with over 95% purity (FIG. 3).

(c) Western immunoblotting with phosphotyrosine monoclonal antibody

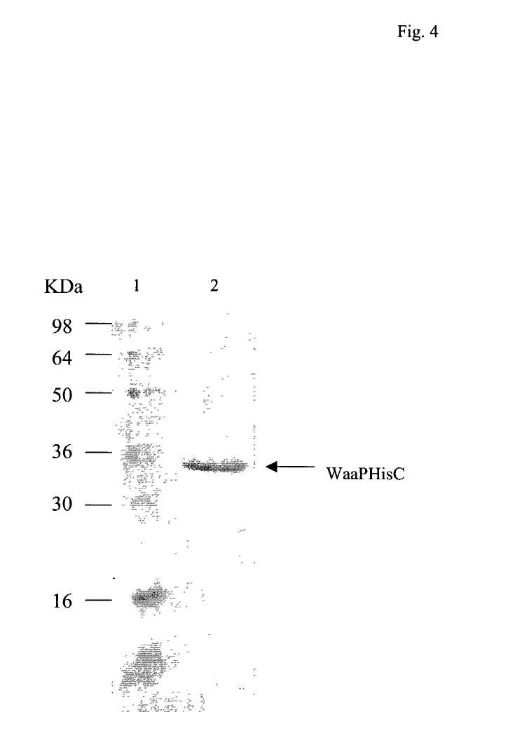

To investigate if WaaP is an autophosphorylated kinase, purified WaaPHisC was examined by Western immunoblotting using phosphotyrosine monoclonal antibody Phosphotyrosine PY-20 (FIG. 4), and a single band was observed in the immunoblotting. This showed that WaaPHisC contains phosphotyrosine residues, which are most likely added by self-phosphorylation. WaaP contains eight tyrosine residues; therefore, the number of tyrosine residues that are phosphorylated needs to identified.

(d) Matrix-assisted laser desorption/ionization time-of-flight (MALDI-TOF) mass spectrometry

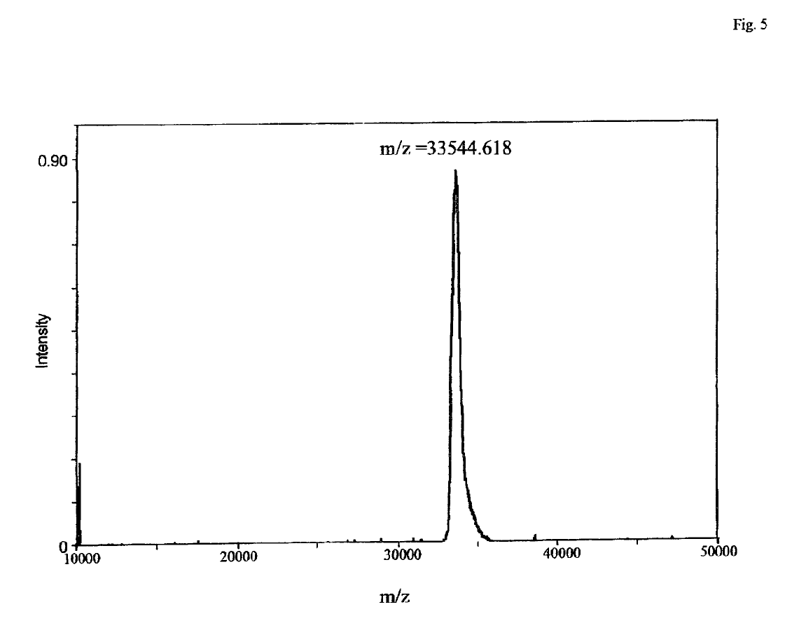

If WaaP protein has been phosphorylated, the mass of the actual protein should be larger than that predicted based on the amino acid sequence. The actual mass of WaaPHisC from the MALDI-TOF analysis was m/z 33544.618 (FIG. 5), which is larger than the predicted (non-phosphorylated) molecular weight of 32897.38. The extra mass 647.328 matched the value of 8.094 units of phosphates substituents (HPO₃, mass=79.969). This result provided the evidence that all the eight tyrosine residues in WaaP may be phosphorylated. This prediction has been further confirmed by peptide mapping with the protease digested WaaPHisC as described following.

(e) Proteolytic peptide mapping—Seven out of eight tyrosine residues were proved to contain phosphate groups.

### Example 2

### ELISA development

(a) Characterization of HF-LPS and mAb 7-4 monoclonal antibody

WaaP was previously shown to be a heptose kinase involved in the phosphorylation of HepI in the core oligosaccharide of P. aeruginosa (Walsh, 2000). To develop an ELISA-based, non-radio-labeling assay to measure the enzymatic activity of WaaP, it was necessary to use the non-phosphorylated LPS as the substrate. In addition, we also need to verify that a previously described inner-core specific monoclonal antibody, mAb 7-4, is specific for the phosphorylated form of P. aeruginosa LPS. Since heptose is not commercially available to serve as the substrate, dephosphorylation of PAO1-LPS was performed by the treatment with 48% hydrofluoric acid (HF) (Kondo, 1992).

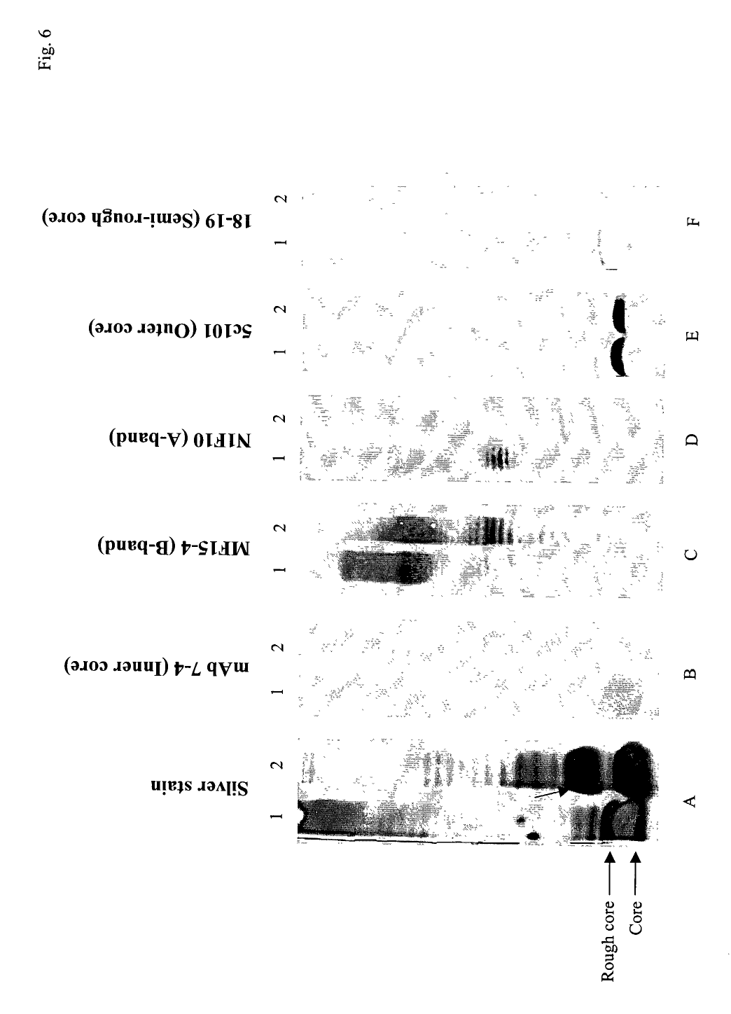

As shown on the silver-stained SDS-PAGE gel (FIG. 6, A), the core (indicated by the arrow) of both HF-LPS and PAO1-LPS migrated similarly except that HF-LPS migrated slightly faster (FIG. 6). This indicated that no sugar residues (obvious molecular mass) were cleaved off from the core region of PAO1-LPS after the HF treatment, and the slightly smaller size of HF-LPS may only be a result from the cleavage of phosphates from the LPS. Interestingly, from the Western immunoblotting analysis, no reaction could be observed between mAb 7-4 and the dephosphorylated LPS (HF-LPS). In contrast, mAb 7-4 reacted strongly with the core LPS of untreated PAO1-LPS. Since HF treatment specifically removes the phosphates from LPS (Katzenellenbogen, 1998; Toukach, 1996), the loss of the recognition of HF-LPS by mAb 7-4 implied that the epitope for the mAb 7-4 is the phosphates in the inner core oligosaccharide. With this valuable finding, we conclude that mAb 7-4 could be used for the development of a non-isotopic enzyme assay i.e. ELISA to quantify the reconstitution of the phosphate(s) on the HF-LPS by the kinase WaaP.

Analyzing the HF-LPS and PAO1-LPS by SDS-PAGE (FIG. 6, A, Lane 2) and Western immunoblotting showed that HF-treatment of LPS did not affect the reactivity of LPS with mAb MF15-4 (B-band specific) (FIG. 6C, Lane 2), however, the B-band in HF-LPS has been partially degraded so that the bands on the gel shifted towards lower molecular mass. Western immunoblotting with mAb N1F10 (A-band specific, FIG. 6, D, Lane 2) showed that A-band LPS has also been degraded to smaller molecules that can no long be recognized by N1F10. As a result of these partial degradations, a new group of oligosaccharide appeared as a thick band (indicated by arrow on the graph). Its relative mobility in the SDS-PAGE gel corresponded to those of core plus two or three O-antigen units, which is smaller than B-band and larger than the semi-rough core (core+1 sugar unit). This band can only be recognized by the outer core specific antibody, mAb 5c101 (FIG. 6, E, Lane 2), indicating that outer core oligosaccharide region was intact. In addition, the semi-rough LPS (core+1 O-antigen unit) had also been partially degraded (FIG. 6, E, Lane 2) as being seen on the silver-stained gel.

(b) Characterization of HF-LPS by critical aggregation concentration

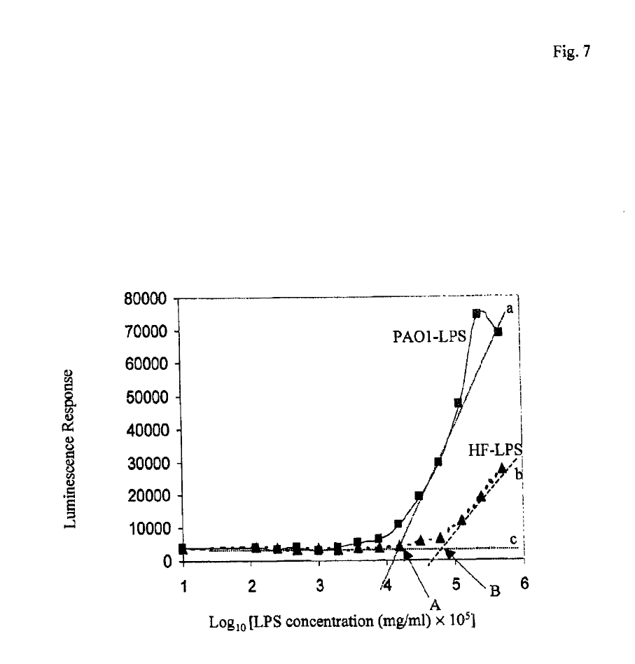

HF-LPS was also characterized on the critical aggregation concentration (CAC) and compared to wild type PAO1-LPS (FIG. 7). The CAC for HF-LPS was calculated to be 0.5 mg·ml⁻¹ that is higher than 0.29 mg·ml⁻¹ for PAO1-LPS. The higher CAC value for HF-LPS implied that it has a lower tendency to form aggregates than PAO1-LPS.

(c) Phosphate analysis on HF-LPS

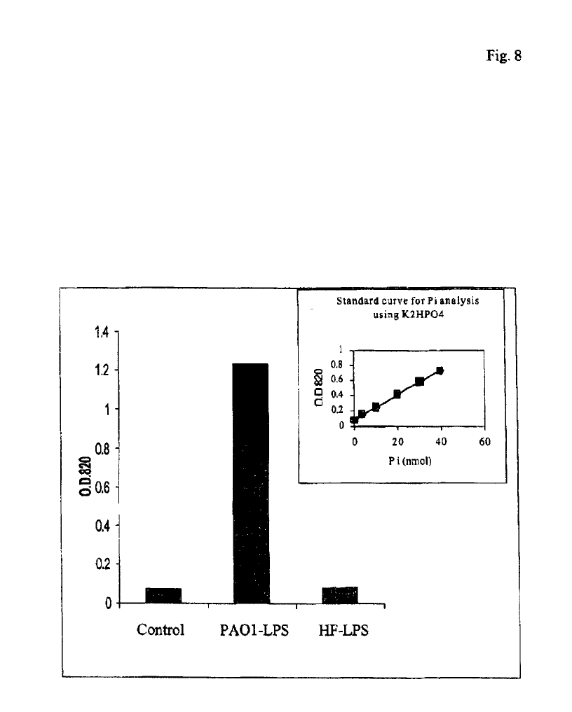

To further verify the dephosphorylation of LPS by HF, phosphate analysis was performed on HF-LPS and compared to that of PAO1-LPS. As was shown in FIG. 8, no phosphate could be detected from HF-LPS compared to 2.18 μg (0.87%, approximately) of phosphate detected from 250 μg PAO1-LPS. This result directly demonstrated that the complete dephosphorylation of HF-LPS was achieved.

(d) Comparison of calorimetric and chemiluminescence based developing method

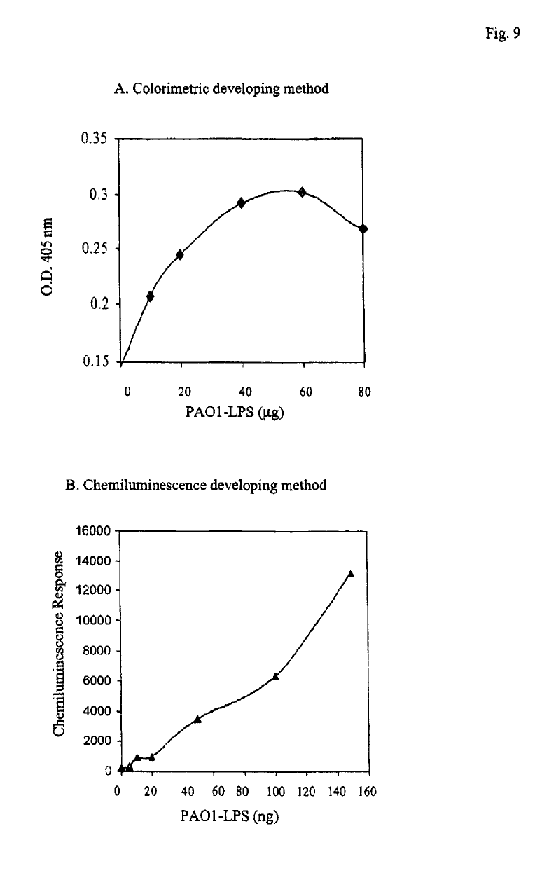

LPS is a huge molecule compared to the few phosphate(s) on the HepI which is the major epitope recognized by mAb 7-4, therefore, a highly sensitive method is required to detect the existence of the phosphates. To increase sensitivity, ELISA was developed to monitor the chemiluminescence signal by using alkaline phosphatase substrate, CDP*. The results of this assay were used to compare with that of the conventional colorimetric ELISA performed using p-nitrophenyl phosphate (pNPP) as the substrate. Both methods gave linear relations of the Response with the LPS amount (FIG. 9). In the chemiluminescence method, as low as 5 nanograms of LPS was sufficient to provide a positive reaction of at least 200 chemiluminescence units above the blank (FIG. 9 B). In contrast, the colorimetric method only allowed the detection of 50 μg with the highest absorbance of 0.3 units at 405 nm (FIG. 9 A). Therefore, the sensitivity of chemiluminescence method was at least 1,000 times higher as compared to the calorimetric method. On the other hand, the fact that we could achieve a straight linear curve from both ELISAs indicated that the response (chemiluminescence or A₄₀₅ nm) units measured was dose-dependent relative to the quantities of LPS antigen used to coat the ELISA well. Importantly, it also validated the effectiveness of the antigen coating method.

(e) Comparison of the incubation of antibodies simultaneously and consecutively

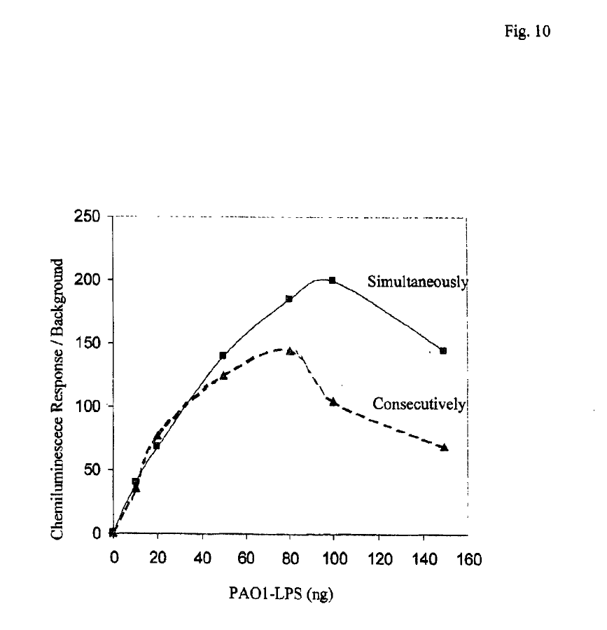

In conventional procedures of ELISA, the incubations with the primary and secondary antibodies are two separate steps with one vigorous washing before adding the secondary antibody. In our studies, we examined the effect of adding the primary and secondary antibodies simultaneously versus adding them consecutively. The results shown in FIG. 10 demonstrated that the simultaneous incubation with the primary and secondary antibodies gave a linear standard curve when 0-80 ng of LPS were used. In contrast, the curve with the consecutive antibody incubations started losing the linearity at 50 ng of LPS. As a further advantage, the simultaneous incubation simplified the ELISA procedure and saved time by removing one washing step (multiple washes) and the subsequent secondary antibody incubation step.

(f) CDP* dilutions

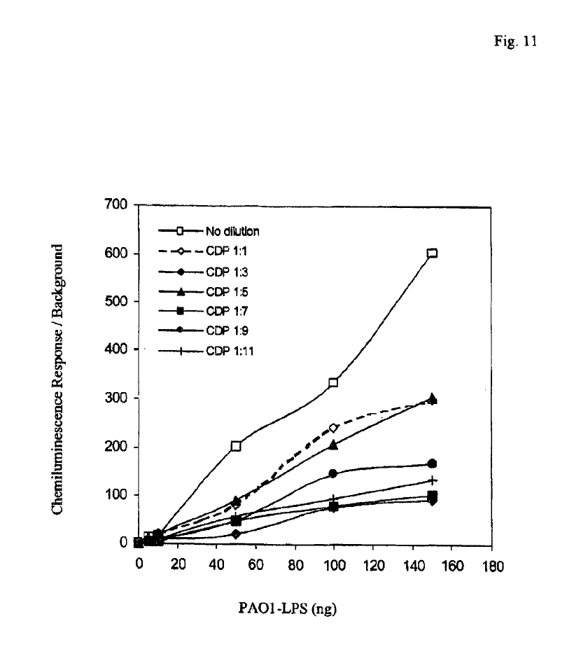

In order to lower the cost of ELISA by saving the developing reagent CDP*, we also performed the ELISAs using different dilutions of CDP* to develop the assays. Results in FIG. 11 demonstrated that the higher concentration of CDP*, the higher Response/Background values indicating the higher sensitivities. With the high cost of screening large numbers of potential inhibitor in mind, it was necessary to lower the cost of the ELISA by choosing the moderate sensitivity, which is sensitive enough to detect nanograms of LPS for our enzyme reaction. Thus, a 1:5 CDP* was used in our following ELISA for enzyme kinetics determination.

(g) Recognition of PAO1-LPS and HF-LPS by mAb 7-4

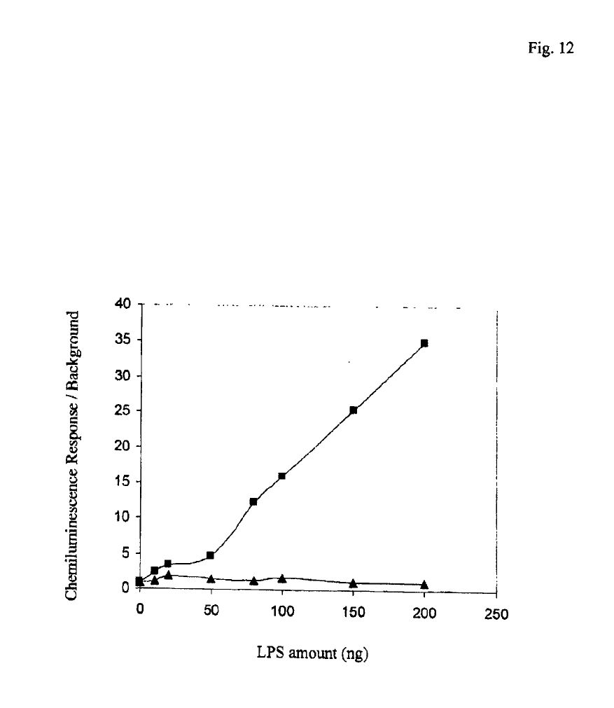

In the Western immunoblotting with mAb 7-4 and the phosphate analysis, we have already identified that mAb 7-4 did not react with HF-LPS. Since chemiluminescence-based ELISA can more sensitively quantify the phosphates in LPS, we performed this ELISA by using up to 200 ng of HF-LPS and compared with that of PAO1-LPS. No positive signal was detected when HF-LPS was used as the antigen (FIG. 12). This further proved that HF-LPS has been completely dephosphorylated.

(h) Kinetics characterization of WaaPHisC using ELISA

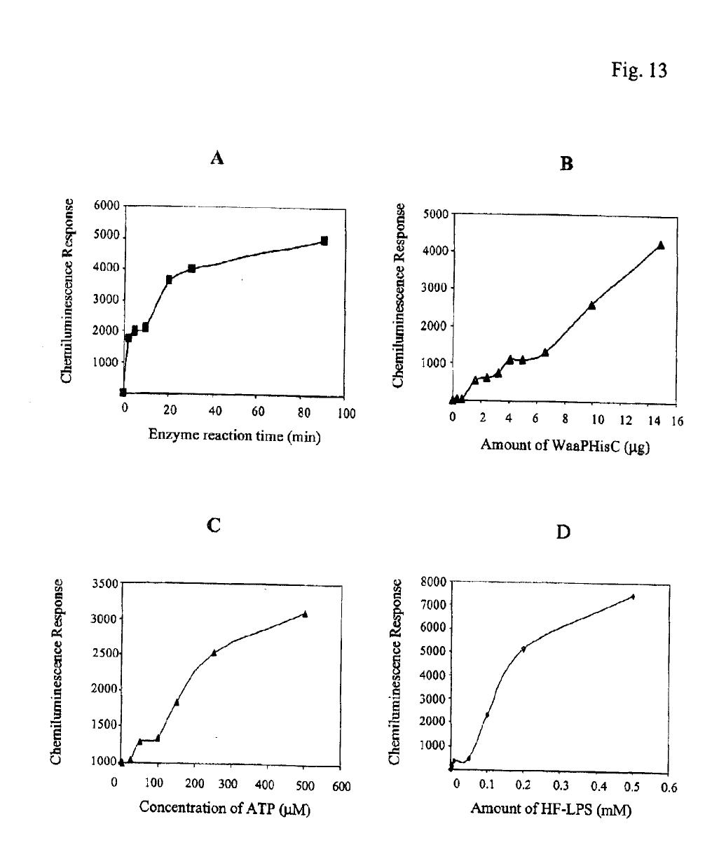

ELISAs on the time course of WaaPHisC reaction (FIG. 13A) showed that the enzyme activities increased sharply in the initial 20 min and slowed down afterward. Therefore, the reactions on the kinetics studies measured the phosphorylation within the initial 15 min.

The enzyme reactions of WaaPHisC were also performed with various concentrations of enzyme (0-15 μg) (FIG. 13B), ATP (0-500 μM) (FIG. 13C) and HF-LPS (0-50 ng) (FIG. 13D), respectively. Our enzyme assays showed that the WaaP-ELISA can be successfully used to quantify the enzyme activity of WaaP in the 96-well microtiter plate. The kinetics parameters were determined based on Michaelis-Menten equations, and the Km was 0.22 mM for ATP and 14.4 μM for HF-LPS. The Vₘₐₓ for the enzyme reaction was 408.24 pmol/min and k_{cat} was 27.23 min⁻¹, about 70% of enzyme activity remained after storage at −20° C. for 7 days.

### Discussion for Examples 1 and 2

Carbohydrates are probably the least understood of all classes of biologically important molecules (Gervay, 1999) and much less is known about the characters of the enzymes i.e. the sugar kinases involved in the synthetic pathway. The tyrosine kinases recently reported (listed in Background) to be involved in the regulation of LPS or capsule biosynthesis in bacteria did not share identities with WaaP nor with the protein kinases. Instead, they shared the Walker A and Walker B consensus among them and in these proteins the tyrosine residues are all localized downstream of Walker B. In contrast, the tyrosine residues in WaaP are scattered throughout the sequence of the protein. But as a similar character with some of these kinases such as Wzc_{ca} in E. coli K-12 (Vincent, 1999; 2000.), Ptk in Acinetobacter johnosoii (Duclos, 1996; Grangeasse, 1998) and Cps D in Streptococcus pneumoniae (Morona, 2000), WaaP is an autophospho-tyrosine kinase.

It is intriguing to observe that WaaP, as a sugar kinase, showed such significant amino acid identities in the functional motifs with the typical protein kinases including members in both protein tyrosine kinase (PTK) and Ser/Thr families. Importantly, this prediction on the conserved motifs was also validated by the site-directed mutagenesis and the following complementation assay. In subdomain I, WaaP had two glycine rich regions, G⁴⁵XG and G⁵⁵XGXG (where X could be any amino acid). Usually the invariant lysine lies at 14 to 23 residues downstream of the conserved glycine, but no mutations have been made to show the importance of the space (Hanks, 1988). Therefore, in WaaP, either of these two glycine regions could be the nucleotide binding site, but regardless of this, the two glycine rich regions provided more hydrophobic environment for ATP binding. The alignment analysis strongly suggested that WaaP is a protein kinase in addition to the sugar kinase. This has also been demonstrated by the characterization of WaaP by Western immunoblotting using phosphotyrosine antibody PY-20, MALDI-TOF mass spectrometry and self-phosphorylation assay. Consequently, it has been demonstrated that WaaP is an auto-phosphorylated tyrosine kinase in which all eight tyrosine residues are phosphorylated. Since WaaP only contains 276 amino acids, the functional domain of kinase, which spanned about 200 amino acids (72%), must be shared by both functions as sugar and protein kinase.

The amino acid sequence of WaaP_{Ec} did not show good alignment among the functional motifs with either WaaP_{Pa} or the protein kinases. Also, in the complementation assay (Table I), the wild type waaP_{Pa} could only partially complement the E. coli F470waaP- and the MIC value to the novobiocin and SDS was higher than in waaP mutant but lower than in the wild type E. coli F470. These implied that WaaP_{Pa} has different characters from WaaP_{Ec} although they have similar function and kinetics characters as heptose kinases. Importantly, the fact that WaaP_{Pa} is also an autophosphotyrosine kinase implied that it might also be involved in other functions i.e. transportation in the LPS biosynthesis like other tyrosine kinases (Vincent, 1999; Duclos, 1996; Grangeasse, 1998; Morona, 2000) other than only as a heptose kinase, or participate in “signaling” in signal transduction and regulation of other bacterial cell functions. This may also be the reason that this enzyme is essential to P. aeruginosa and a waaP mutant was lethal whereas waaP_{Ec} was not.

As a crucial enzyme to P. aeruginosa, WaaP can be a good drug target to develop new antibiotics. In recent years, enormous effort in developing protein tyrosine kinase inhibitors has been made for diseases such as cancer, psoriasis and osteoporosis. Several new high-throughput PTK assay technologies have been described and some inhibitors already have been in clinical trials (Obeidi, 1998).

LPS is a large molecule with a hydrophobic tail of lipid A, it tends to form aggregates in solution. To avoid the effect of the LPS aggregation on the ELISA, the critical aggregation concentrations (CAC) on both HF-LPS and PAO1-LPS were determined. NPN used in this study is a well-known fluorescence marker for its propensity to partition into hydrophobic region of large molecule to give a fluorescence response at the emission wavelength of 425 nm. The results showed that HF-LPS has less potential to form aggregates in solution (Tris-HCl, pH 7.8, the same conditions as those used in enzyme reaction) than PAO1-LPS. The concentrations of both LPS used in ELISA are well below their CACs respectively. Thus, without the concern of the aggregation problems in HF-LPS, the ELISA established using PAO1-LPS could be applied to that of enzyme assay that contains HF-LPS.

For developing an assay to determine the phosphorylation activity of WaaP, LPS without phosphate was generated to serve as the substrate. HF treatment is the standard dephosphorylation method used in structure analysis of core oligosaccharide in LPS (Dasgupta, 1994; Kondo, 1992; Katzenellenbogen, 1998; Toukach, 1996). Kondo et al. (1992) used this method to dephosphorylate the LPS from seven strains from Vibtionaceae, and further revealed the existence of the phosphorylated Kdo by the followed NMR analysis. Structure analysis of LPS using HF treatment followed by NMR analysis was also reported by Katzenellenbogen (Katzenellenbogen, 1998) and Toukach (Toukach, 1996) for the LPS from Hafria alvei. They also reported that HF treatment had the side effect of removing the lateral sugar residue of beta-galactofuranose from the LPS. In the result reported herein, HF also degraded the long chain LPS to some extent. However, no evidence was shown that HF treatment affected the structure of the core oligasaccharide. This is consistent with our result shown in the FIG. 6 that the core of HF-LPS migrated almost the same as PAO1-LPS indicating the intact core in HF-LPS regardless of the removed phosphates as confirmed by the following phosphate analysis. The phosphate analysis method used in this study contains the procedure of using 1 N HCl hydrolysis after the oxidizing and ashing in Mg(NO₃)/Ethanol, and this treatment ensured the fully hydrolysis of the pyrophosphate formed in the ashing (Ames, 1960), and therefore the accuracy of the results.

An advantage of using HF-LPS is the fact that this modified form of LPS contains much more shorter-chain than wild type PAO1-LPS. The much shorter O-antigen chain may provide less steric hindrance for the enzyme to reach the heptose during the enzymatic phosphorylation. The identification of the phosphate as the epitope of mAb 7-4 in this study was critical for the development of the ELISA as the non-radio-labeling assay for WaaP.

Improved Response/Background values using the simultaneous incubation of the antibodies, were also reported by Lehel (Lehel, 1997) in the ELISA using phosphoserine-specific YC10 as the primary antibody to assay protein kinase A and protein kinase C. Using this strategy in the ELISA reported herein (FIG. 10) further simplified and shortened the procedure.

In addition to the advantage of the highly improved sensitivity with chemiluminescence, the incubation step after adding the chemiluminescence substrate needs only 20 min at room temperature compared to the incubation for 2 h at 37° C. to develop the calorimetric signal in the conventional calorimetric ELISA. Another advantage of using chemiluminescence over calorimetric ELISA is high specificity. The chemiluminescence signal was specifically generated from the reaction of the alkaline phosphatase (conjugated to the secondary antibody), exhibiting minimum interference from the solvents and test substances from various sources. This can minimize the occurrence of false-positive results. Therefore, this is a valuable property for the use of the method in high-throughput drug screening.

Another innovative feature of the WaaP-ELISA using mAb 7-4 is the capability to quantitatively determine the enzyme activity of WaaPHisC with high sensitivity. The enzyme activity of WaaP from E. coli was recently reported by Yethon (Yethon, 2001) using the LPS isolated from E. coli waaP mutant as the substrate. For this enzyme, γ-[³³P]ATP was used to assay the enzyme activity, and the Km on ATP was 0.13 mM which is quite close to that determined by us at 0.22 mM for WaaPHisC_{Pa}. In our study, the Km of WaaPHisC_{Pa} for HF-LPS was 14.4 μM comparing to 76 μM for WaaP_{Ec}. While not wishing to be limited by theory, the lower Km_{Pa} may be because the HF-LPS used in this study contains shorter-chain LPS and therefore has better access for the enzyme to reach and phosphorylate the heptose. The Vₘₐₓ of WaaP_{Pa} was 408.24 pmol.min⁻¹ and k_{cat} was 27.23 min⁻¹. However, the k_{cat} of WaaP_{Ec} has not been reported. These two proteins share 52% identities at amino acid level and the differences in the sequences may result in the variations in the catalytic characters. The WaaP_{Ec} reported had the His-tag at the N-terminus of the protein while the WaaP described in this paper contains the C-terminal His-tag.

In conclusion, WaaP_{Pa} has been identified to be a novel autophosphotyrosine kinase in prokaryotes by possessing phosphotyrosine residues in its sequence. It was also shown to be distinct from WaaP_{Ec} although they are both kinases to phosphorylate heptose of LPS in P. aeruginosa and E. coli, respectively. Moreover, a sensitive ELISA method, a non-radio-labeling assay, was developed for the enzyme assay of WaaP and it was successfully applied to the kinetics studies of WaaP. Thus, this assay method can be used for screening of novel antimicrobial compounds against infection from P. aeruginosa and a host of other Gram-nagative bacteria.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### FULL CITATIONS FOR REFERENCES REFERRED TO IN THE SPECIFICATION

al-Obeidi, F. A., Wu, J. J., and Lam, K. S. (1998) Protein tyrosine kinases: structure, substrate specificity, and drug discovery. Biopolymers 47: 197-223.

Altschul, S. F., Madden, T. L., Schaffer, A. A., Zhang, J., Zhang, Z., Miller, W., and Lipman, D. J. (1997) Gapped BLAST and PSI-BLAST: a new generation of protein database search programs. Nucleic Acids Res 25: 3389-3402.

Ames, B. N. and Dubin, D. T. (1960) The role of polyamines in the neutralization of bacteriophate deoxyribonucleic acid. J Biol Chem 235: 769-775

Anderson, S. K., Gibbs, C. P., Tanaka, A., Kung, H. J., and Fujita, D. J. (1985) Human cellular src gene: nucleotide sequence and derived amino acid sequence of the region coding for the carboxy-terminal two-thirds of pp60c-src. Mol Cell Biol 5: 1122-1129.

Bantroch, S., Buhler, T., and Lam, J. S. (1994) Appropriate coating methods and other conditions for enzyme-linked immunosorbent assay of smooth, rough, and neutral lipopolysaccharides of Pseudomonas aeruginosa. Clin Diagn Lab Immunol 1: 55-62.

Braunwalder, A. F., Yarwood, D. R., Sills, M. A., and Lipson, K. E. (1996) Measurement of the protein tyrosine kinase activity of c-src using time-resolved fluorometry of europium chelates. Anal Biochem 238: 159-164.

Brenner, S. (1987) Phosphotransferase sequence homology. Nature 329: 21.

Burnette, W. N. (1981) “Western blotting”: electrophoretic transfer of proteins from sodium dodecyl sulfate--polyacrylamide gels to unmodified nitrocellulose and radiographic detection with antibody and radioiodinated protein A. Anal Biochem 112: 195-203.

Celenza, J. L., and Carlson, M. (1986) A yeast gene that is essential for release from glucose repression encodes a protein kinase. Science 233: 1175-1180.

Cozzone, A. J. (1998) Post-translational modification of proteins by reversible phosphorylation in prokaryotes. Biochimie 80: 43-48.

Dasgupta, T., de Kievit, T. R., Masoud, H., Altman, E., Richards, J. C., Sadovskaya, I., Speert, D. P., and Lam, J. S. (1994) Characterization of lipopolysaccharide-deficient mutants of Pseudomonas aeruginosa derived from serotypes O3, O5, and O6. Infect Immun 62: 809-817.

de Kievit, T. R., and Lam, J. S. (1994) Monoclonal antibodies that distinguish inner core, outer core, and lipid A regions of Pseudomonas aeruginosa lipopolysaccharide. J Bacteriol 176: 7129-7139.

de Kievit, T. R., Dasgupta, T., Schweizer, H., and Lam, J. S. (1995) Molecular cloning and characterization of the rfc gene of Pseudomonas aeruginosa (serotype O5). Mol Microbiol 16: 565-574.

de Kievit, T. R., and J. S. Lam. (1997) Isolation and characterization of two genes, rfac and rfaF, involved in Pseudomonas aeruginosa serotype O5 inner core biosynthesis. J. Bacteriol. 179:3451-3457.

Duclos, B., Grangeasse, C., Vaganay, E., Riberty, M., and Cozzone, A. J. (1996) Autophosphorylation of a bacterial protein at tyrosine. J Mol Biol 259: 891-895.

Fomsgaard, A., Freudenberg, M. A., and Galanos, C. (1990) Modification of the silver staining technique to detect lipopolysaccharide in polyacrylamide gels. J Clin Microbiol 28: 2627-2631.

Garcia, P., Shoelson, S. E., George, S. T., Hinds, D. A., Goldberg, A. R., and Miller, W. T. (1993) Phosphorylation of synthetic peptides containing Tyr-Met-X-Met motifs by nonreceptor tyrosine kinases in vitro. J Biol Chem 268: 25146-25151.

Gervay, J., and McReynolds, K. D. (1999) Utilization of ELISA technology to measure biological activities of carbohydrates relevant in disease status. Curr Med Chem 6: 129-153.

Grangeasse, C., Doublet, P., Vincent, C., Vaganay, E., Riberty, M., Duclos, B., and Cozzone, A. J. (1998) Functional characterization of the low-molecular-mass phosphotyrosine- protein phosphatase of Acinetobacter johnsonii. J Mol Biol 278: 339-347.

Hanks, S. K., Quinn, A. M., and Hunter, T. (1988) The protein kinase family: conserved features and deduced phylogeny of the catalytic domains. Science 241: 42-52.

Hanks, S. K., and Quinn, A. M. (1991) Protein kinase catalytic domain sequence database: identification of conserved features of primary structure and classification of family members. Methods Enzymol 200: 38-62.

Helander, I. M,. Vaara, M., Sukupolvi, S., Rhen, M., Saarela, S., Zähringer, U., and Mäkelä, P. H. (1989) rfaP mutants of Salmonella typhimurium. Eur. J. Biochiem. 185: 541-546.

Horton, R. M., Ho, S. N., Pullen, J. K., Hunt, H. D., Cai, Z., and Pease, L. R. (1993) Gene splicing by overlap extension. Methods Enzymol 217: 270-279.

Huff, J. P., Grant, B. J., Penning, C. A., and Sullivan, K. F. (1990) Optimization of routine transformation of Escherichia coli with plasmid DNA. Biotechniques 9: 570-572, 574, 576-577.

Ilan, O., Bloch, Y., Frankel, G., Ullrich, H., Geider, K., and Rosenshine, I. (1999) Protein tyrosine kinases in bacterial pathogens are associated with virulence and production of exopolysaccharide. EMBO J 18: 3241-3248.

Kamps, M. P., and Sefton, B. M. (1986) Neither arginine nor histidine can carry out the function of lysine-295 in the ATP-binding site of p60src. Mol Cell Biol 6: 751-757.

Katzenellenbogen, E., Kocharova, N. A., Zatonsky, G. V., Mieszala, M., Gamian, A., Bogulska, M., Shashkov, A. S., Romanowska, E., and Knirel, Y. A. (1998) Immunochemical studies of the lipopolysaccharide O-specific polysaccharide of Hafnia alvei PCM 1199 related to H. alvei PCM 1205. Eur J Biochem 251: 980-985.

Kondo, S., Haishima, Y., and Hisatsune, K. (1992) Taxonomic implication of the apparent undetectability of 3-deoxy-D-manno-2-octulosonate (Kdo) in lipopolysaccharides of the representatives of the family Vibrionaceae and the occurrence of Kdo 4—phosphate in their inner-core regions. Carbohydr Res 231: 55-64.

Laemmli, U. K. (1970) Cleavage of structural proteins during the assembly of the head of bacteriophage T4. Nature 227: 680-685.

Lehel, C., Daniel-Issakani, S., Brasseur, M., and Strulovici, B. (1997) A chemiluminescent microtiter plate assay for sensitive detection of protein kinase activity. Anal Biochem 244: 340-346.

Madhusudan, Trafny, E. A., Xuong, N. H., Adams, J. A., Ten Eyck, L. F., Taylor, S. S., and Sowadski, J. M. (1994) cAMP-dependent protein kinase: crystallographic insights into substrate recognition and phosphotransfer. Protein Sci 3: 176-187.

Morona, J. K., Paton, J. C., Miller, D. C., and Morona, R. (2000) Tyrosine phosphorylation of CpsD negatively regulates capsular polysaccharide biosynthesis in Streptococcus pneunioniae. Mol Microbiol 35: 1431-1442.

Parker, P. J., Coussens, L., Totty, N., Rhee, L., Young, S., Chen, E., Stabel, S., Waterfield, M. D., and Ullrich, A. (1986) The complete primary structure of protein kinase C—the major phorbol ester receptor. Science 233: 853-859.

Sadovskaya, I., Brisson, J. R., Lam, J. S., Richards, J. C., and Altman, E. (1998) Structural elucidation of the lipopolysaccharide core regions of the wild-type strain PAO1 and O-chain-deficient mutant strains AK1401 and AK1012 from Pseudomonas aeruginosa serotype O5. Eur J Biochem 255: 673-684.

Sambrook, J., Fritsch, E. F., Maniatis, T. (1989) Molecular cloning: a laboratory Manual, 2nd Ed., Cold Spring Harbor Laboratory, Cold Spring Harbor, (N.Y.) A2

Schraag, B., Staal, G. E., Adriaansen-Slot, S. S., Salden, M., and Rijksen, G. (1993) Standardization of an enzyme-linked immunosorbent assay for the determination of protein tyrosine kinase activity. Anal Biochem 211: 233-239.

Smith, P. K., Krohn, R. I., Hermanson, G. T., Mallia, A. K., Gartner, F. H., Provenzano, M. D., Fujimoto, E. K., Goeke, N. M., Olson, B. J., and Klenk, D. C. (1985) Measurement of protein using bicinchoninic acid. Anal Biochem 150: 76-85.

Toukach, F. V., Shashkov, A. S., Katzenellenbogen, E., Kocharova, N. A., Czarny, A., Knirel, Y. A., Romanowska, E., and Kochetkov, N. K. (1996) Structure of the O-specific polysaccharide of Hafnia alvei PCM 1222 containing 2-aminoethyl phosphate. Carbohydr Res 295: 117-126.

Ullrich, A., Coussens, L., Hayflick, J. S., Dull, T. J., Gray, A., Tam, A. W., Lee, J., Yarden, Y., Libermann, T. A., Schlessinger, J., and et al. (1984) Human epidermal growth factor receptor cDNA sequence and aberrant expression of the amplified gene in A431 epidermoid carcinoma cells. Nature 309: 418-425.

Vincent, C., Doublet, P., Grangeasse, C., Vaganay, E., Cozzone, A. J., and Duclos, B. (1999) Cells of Escherichia coli contain a protein-tyrosine kinase, Wzc, and a phosphotyrosine-protein phosphatase, Wzb. J Bacteriol 181: 3472-3477.

Vincent, C., Duclos, B., Grangeasse, C., Vaganay, E., Riberty, M., Cozzone, A. J., and Doublet, P. (2000) Relationship between exopolysaccharide production and protein-tyrosine phosphorylation in gram-negative bacteria. J Mol Biol 304: 311-321.

Walsh, A. G., Matewish, M. J., Burrows, L. L., Monteiro, M. A., Perry, M. B., and Lam, J. S. (2000) Lipopolysaccharide core phosphates are required for viability and intrinsic drug resistance in Pseudomonas aeruginosa. Mol Microbiol 35: 718-727.

West, S. E., Sample, A. K., and Runyen-Janecky, L. J. (1994) The vfr gene product, required for Pseudomonas aeruginosa exotoxin A and protease production, belongs to the cyclic AMP receptor protein family. J Bacteriol 176: 7532-7542.

Westphal, O., and Jann, K. (1965) Bacterial lipopolysaccharide. Extraction with phenol-hot water and further applications of the procedure. Methods Carbohydr Chem 5: 83-91

Wierenga, R. K., and Hol, W. G. (1983) Predicted nucleotide-binding properties of p21 protein and its cancer-associated variant. Nature 302: 842-844.

Wugeditsch, T., Paiment, A., Hocking, J., Drummelsmith, J., Forrester, C., and Whitfield, C. (2000) Phosphorylation of Wzc, a tyrosine autokinase, is essential for assembly of group 1 capsular polysaccharides in Escherichia coli. J Biol Chem 26: 26.

Yethon, J. A., Heinrichs, D. E., Monteiro, M. A., Perry, M. B., and Whitfield, C. (1998) Involvement of waaY, waaQ, and waaP in the modification of Escherichia coli lipopolysaccharide and their role in the formation of a stable outer membrane. J Biol Chem 273: 26310-26316.

Yethon, J. A., and Whitfield, C. (2001) Purification and characterization of WaaP from Escherichia coli, a lipopolysaccharide kinase essential for outer membrane stability. J Biol Chem 276: 5498-5504.

Zhang, C. C. (1996) Bacterial signalling involving eukaryotic-type protein kinases. Mol Microbiol 20: 9-15.

Zhao, X., and Lam, J. S. (2002) WaaP of Pseudomonas aeruginosa is a novel eukaryotic type protein-tyrosine kinase as well as a sugar kinase essential for the biosynthesis of core lipopolysaccharide. J Biol Chem 277:4722-4730.

Zhao, X., Wenzel, C. Q. and Lam, J. S. (2002) Development of a non-radiolabeling assay for WaaP, an essential sugar kinase involved in the biosynthesis of core lipopolysaccharide of Pseudomonas aeruginosa. Antimicrob. Agents Chemother. 46:2035-2037.

We claim:

1. A method for assaying for modulators of an enzyme involved in the phosphorylation of the inner core oligosaccharide of lipopolysaccharide (LPS), comprising the steps of:
(a) incubating a test sample comprising (i) the enzyme, (ii) a candidate substance; and (iii) substrates comprising dephosphorylated LPS and a source of phosphate;
(b) adding to the test sample at least one antibody comprising an antibody that binds to phosphorylated LPS while not binding to dephosphorylated LPS; and
(c) detecting phosphorylated LPS in the test sample by measuring the binding of the at least one antibody to phosphorylated LPS, wherein an increase or decrease in the amount of phosphorylated LPS in the test sample in the presence of the candidate substance indicates that the candidate substance is a modulator.

2. The method according to claim 1, wherein the antibody comprises mAb 7-4.

3. The method according to claim 1, wherein the dephosphorylated LPS comprises HF-LPS.

4. The method according to claim 1, wherein the enzyme is selected from the group consisting of E. coli WaaP, Gram-negative bacteria WaaP, P. aeruginosa WaaP and S. typhimurium WaaP.

5. The method according to claim 4, wherein the enzyme comprises P. aeruginosa WaaP.

6. The method according to claim 1, wherein the source of phosphate is adenosine triphosphate (ATP).

7. The method according to claim 1, wherein the amount of phosphorylated LPS is quantified using enzyme-linked immunosorbent assay (ELISA).

8. The method according to claim 7, wherein the ELISA is developed using a method selected from chemiluminescence and colorimetrics.

9. The method according to claim 8, wherein the ELISA is developed using chemiluminescence.

10. The method according to claim 1, wherein step (c) is:
(c) adding at least two antibodies selected from the group consisting of an antibody that binds to phosphorylated LPS while not binding to dephosphorylated LPS and alkaline phosphatase conjugated-goat anti-mouse F(ab′)₂.

11. The method according to claim 10, wherein the two antibodies are added simultaneously.

12. A method for assaying for inhibitors of an enzyme involved in the phosphorylation of the inner core oligosaccharide of LPS, comprising the steps of:
(a) incubating a test sample comprising (i) the enzyme, (ii) a candidate substance; and (iii) substrates comprising dephosphorylated LPS and a source of phosphate;
(b) adding to the test sample at least one antibody that binds to phosphorylated LPS while not binding to dephosphorylated LPS; and
(c) detecting phosphorylated LPS in the test sample by measuring the binding of the at least one antibody to phosphorylated LPS, wherein a decrease in the amount of phosphorylated LPS in the test sample in the presence of the candidate substance indicates that the candidate substance is an inhibitor.

13. The method according to claim 10, wherein the antibody that binds to phosphorylated LPS while not binding to dephosphorylated LPS comprises mAb 7-4.

14. The method according to claim 12, wherein the dephosphorylated LPS comprises HF-LPS.

15. The method according to claim 12, wherein the enzyme is selected from the group consisting of E. coli WaaP, Gram-negative bacteria WaaP, P. aeruginosa WaaP and S. typhimurium WaaP.

16. The method according to claim 15, wherein the enzyme comprises P. aeruginosa WaaP.

17. The method according to claim 12-, wherein the source of phosphate comprises ATP.

18. The method according to claim 12, wherein the amount of phosphorylated LPS is quantified using ELISA.

19. The method according to claim 18, wherein the ELISA is developed using a method selected from chemiluminescence and colorimetrics.

20. The method according to claim 19, wherein the ELISA is developed using chemiluminescence.

21. The method according to claim 12, wherein step (c) is:
(c) adding at least two antibodies selected from the group consisting of an antibody that binds to phosphorylated LPS while not binding to dephosphorylated LPS and alkaline phosphatase conjugated-goat anti-mouse F(ab′)₂.

22. The method according to claim 21 wherein the antibody that binds to phosphorylated LPS while not binding to dephosphorylated LPS comprises mAb 7-4.

23. The method according to claim 21, wherein the two antibodies are added simultaneously.

24. A kit comprising:
(a) reagents for performing an enzyme reaction, including an aliquot of dephosphorylated LPS, an aliquot of a source of phosphate, and an aliquot of an enzyme involved in the phosphorylation of the inner core oligosaccharide of LPS; and
(b) reagents for performing an ELISA, including an aliquot of an antibody that binds to phosphorylated LPS while not binding to dephosphorylated LPS and an aliquot of the secondary antibody.

25. The kit according to claim 24, wherein the antibody that binds phosphorylated LPS while not binding dephosphorylated LPS comprises mAb 7-4.

26. The kit according to claim 24, wherein the dephosphorylated LPS comprises HF-LPS.

27. The kit according to claim 24, wherein the source of phosphate comprises ATP.

28. The kit according to claim 24, wherein the enzyme involved in the phosphorylation of the inner core oligosaccharide of LPS is selected from the group consisting of E. coli WaaP, Gram negative bacteria WaaP, P. aeruginosa WaaP and S. typhimurium WaaP.

29. The kit according to claim 28, wherein the enzyme involved in the phosphorylation of the inner core oligosaccharide of LPS comprises P. aeruginosa WaaP.

30. The kit according to claim 24, wherein the reagents for performing an ELISA further comprises an aliquot of alkaline phosphatase conjugated-goat anti-mouse F(ab′)₂.

31. The kit according to claim 24, further comprising printed instructions.

32. A method of conducting a target discovery business comprising:
(a) providing one or more assay systems for identifying agents by their ability to modulate an enzyme involved in the phosphorylation of the inner core oligosaccharide of LPS, said assay systems using a method of the invention;
(b) conducting therapeutic profiling of agents identified in step (a) for efficacy and toxicity in animals; and
(c) licensing, to a third party, the rights for further drug development and/or sales or agents identified in step (a), or analogs thereof.

33. The use of a method according to claim 1 to screen for modulators of an enzyme involved in the phosphorylation of the inner core oligosaccharide of LPS.

34. The use according to claim 33, wherein the modulator is an inhibitor of an enzyme involved in the phosphorylation of the inner core oligosaccharide of LPS.

35. A modulator identified with the method of claim 1 or an inhibitor identified with the method of claim 12.
